(19) United States
(12) Patent Application Publication
(10) Pub. No.: US 2020/0010344 A1
(43) Pub. Date: Jan. 9, 2020

(54) SHIP EQUIPMENT MANAGEMENT SYSTEM, EQUIPMENT MANAGEMENT PROGRAM, AND EQUIPMENT MANAGEMENT METHOD

(71) Applicant: KURITA WATER INDUSTRIES LTD., Tokyo (JP)

(72) Inventors: Kotaro FUKUZAWA, Tokyo (JP); Kaname HARADA, Tokyo (JP); Kazuhiko TSUNODA, Tokyo (JP); Minoru YAGI, Tokyo (JP)

(21) Appl. No.: 16/467,711

(22) PCT Filed: Sep. 15, 2017
(86) PCT No.: PCT/JP2017/033507

§ 371 Date: Jun. 7, 2019

(30) Foreign Application Priority Data
Dec. 12, 2016 (JP) 2016-240443

(51) Int. Cl. C02F 1/76; C02F 1/50; B63B 13/00; B63J 4/00; G01N 33/18

(52) U.S. Cl. CPC C02F 1/76; C02F 1/50; C02F 2103/008; B63J 4/002; G01N 33/1826; B63B 13/00

(57) ABSTRACT

An equipment management system for a ship includes a communicating part that acquires from the ship by radio notification information including one piece of state information, or two or more pieces of state information selected from pieces of state information on at least ballast water process equipment, boiler equipment, and water desalination process equipment loaded on the ship, and a processing part that extracts the pieces of state information on the ballast water process equipment, the boiler equipment, or the water desalination process equipment from the notification information acquired from the ship, produces management information including drug information at least necessary for the ballast water process equipment, the boiler equipment, or the water desalination process equipment, and causes the communicating part to send out the management information to the ship sending out at least the notification information.

### TECHNICAL FIELD

The present invention relates to a management technique used for remote management of various types of equipment including pieces of water process equipment such as ballast water process equipment, boiler equipment, and water desalination process equipment, and environment-related equipment that are loaded on a ship.

### BACKGROUND ART

A ship has various types of equipment loaded thereon such as ballast water process equipment, boiler equipment, and water desalination process equipment. For example, a measure is taken for a tanker according to which the ballast water loaded thereon is discharged at a harbor of an anchorage site thereof and crude oil or the like is loaded thereon. The ballast water loaded thereon at the departure site includes microorganisms of the departure site and it is necessary to execute disinfection process for the ballast water underway or at the anchorage site and to discharge the ballast water in the state where the ballast water complies with the standards of the anchorage site.

Concerning this ballast water process, it is known that the ballast water needs to be processed to achieve the water management standards determined by International Maritime Organization (IMO) (for example, Patent Document 1).

### PRIOR ART DOCUMENT

### Patent Document

Patent Document 1: Japanese Laid-Open Patent Publication No. 2009-112978

### SUMMARY OF THE INVENTION

### Problem to be Solved by the Invention

A ship has various types of equipment such as ballast water process equipment, boiler equipment, and water desalination process equipment loaded thereon, and at least the ballast water process equipment, the boiler equipment, the water desalination process equipment, and the like relate to water processes, and a process of inputting a drug to clean up the water, and the like is necessary.

To execute the management of the pieces of equipment such as the ballast water process equipment, the boiler equipment, and the water desalination process equipment not only by the ship but also by the land such as a management center, it is necessary to notify the land of pieces of state information and the like acquired from the ballast water process equipment, the boiler equipment, or the water desalination process equipment separately from each other and to separately execute the management for the ship from the land. Even for one single ship, this type of management needs considerable labor and time and, when many ships interspersed around the world are to be managed, the management becomes complicated and a problem arises that no quick response can be made. When no proper response can be made, loading of crude oil and the like are obstructed such as restrictions imposed on the discharge of the ballast water. Even in the case where the disinfection process for the ballast water is executed underway, when the necessary drugs run out, the process is delayed or the like, the run-out drugs need to be procured at the anchorage site, and exchange of information is also necessary between the ship and the management center for the determination as to which drugs are necessary. The similar management is also necessary for not only the ballast water but also the other pieces of equipment such as the boiler equipment and the water desalination process equipment. This type of management is an important item that relates also to safe sailing of the ship.

In view of the above problem, an object of the present invention is to realize management of one piece of state information or a combination of plural pieces of state information, or collective management of the pieces of state information selected from pieces of state information that are acquired from at least the ballast water process equipment, the boiler equipment, and the water desalination process equipment.

### Means for Solving Problem

To achieve the object, according to an aspect of an equipment management system for a ship of the present invention, the ship equipment management system for the ship only has to include a communicating part that acquires from a ship by radio notification information including one piece of state information, or two or more pieces of state information selected from pieces of state information on at least ballast water process equipment, boiler equipment, and water desalination process equipment loaded on the ship, and a processing part that extracts the pieces of state information on the ballast water process equipment, the boiler equipment, or the water desalination process equipment from the notification information acquired from the ship, produces management information including drug information at least necessary for the ballast water process equipment, the boiler equipment, or the water desalination process equipment, and causes the communicating part to send out the management information to the ship sending out at least the notification information.

In the equipment management system for the ship, the processing part may cause the communicating part to send out the management information to an owner or a manager of the ship that sends out the notification information.

In the equipment management system for the ship, the management information may further include reporting document information to be used for a journal information or a ballast process report.

In the equipment management system for the ship, the processing part may manage remaining amounts of drugs necessary for the pieces of equipment of the ballast water process equipment, the boiler equipment, and the water desalination process equipment, and may arrange any one of the drugs at an anchorage site of the ship in accordance with the remaining amount of the drug.

To achieve the objective, according to an aspect of the equipment management system for the ship of the present invention, the equipment management system for the ship only has to include a processing part that produces notification information including one piece of state information, or two or more pieces of state information selected from pieces of state information on at least ballast water process equipment, boiler equipment, and water desalination process equipment loaded on a ship, and a communicating part that transmits by radio the notification information produced by the processing part.

In the equipment management system for the ship, the communicating part may receive by radio management information including drug information at least necessary for the ballast water process equipment, the boiler equipment, or the water desalination process equipment, and may send out the management information to the processing part, and the processing part may present the management information.

To achieve the objective, according to an aspect of an equipment management program for a ship of the present invention, the equipment management program for the ship is an equipment management program executed by a computer, and the computer implements a function of producing notification information that includes one piece of state information, or two or more pieces of state information selected from pieces of state information on at least ballast water process equipment, boiler equipment, and water desalination process equipment loaded on a ship, a function of extracting the pieces of state information on the ballast water process equipment, the boiler equipment, or the water desalination process equipment from the notification information and producing management information that includes drug information necessary for the ballast water process equipment, the boiler equipment, or the water desalination process equipment, and a function of sending out the notification information or the management information.

To achieve the objective, according to an aspect of an equipment management method for a ship of the present invention, the equipment management method for the ship only has to include producing notification information that includes one piece of state information, or two or more pieces of state information selected from pieces of state information on at least ballast water process equipment, boiler equipment, and water desalination process equipment loaded on a ship, extracting the pieces of state information on the ballast water process equipment, the boiler equipment, or the water desalination process equipment from the notification information and producing management information that includes drug information necessary for the ballast water process equipment, the boiler equipment, or the water desalination process equipment, and sending out the notification information or the management information.

### Effect of the Invention

According to the present invention, any one of the following effects is achieved.

(1) A management center away from a ship can timely monitor the states of the ballast water process equipment, the boiler equipment, and the water desalination process equipment, from the pieces of state information acquired from at least the ballast water process equipment, the boiler equipment, and the water desalination process equipment included in the notification information from the ship sailing in a distant place.

(2) Any necessary management information can be provided from the management center to the ship, and the ship can quickly take any measure based on the management information.

Other objects, features, and advantages of the present invention will become more apparent when reading the embodiments and Examples herein with reference to the accompanying drawings.

### BRIEF DESCRIPTION OF DRAWINGS

FIG. 1 is a diagram of an example of an equipment management system for a ship according to an embodiment.

FIG. 2 is a diagram of a process sequence for the ship.

FIG. 3A is a diagram of a process sequence for a management center and FIG. 3B is a diagram of a process sequence for the ship.

FIG. 4 is a diagram of an example of the equipment management system for the ship according to Example 1.

FIG. 5 is a diagram of an example of equipment information.

FIG. 6 are diagrams each of an example of ship/equipment data.

FIG. 7 is a diagram of an example of management/process data.

FIG. 8 is a diagram of an example of the hardware configuration of a processing part.

FIG. 9 is a diagram of an example of the hardware configuration of a management server.

FIG. 10 is a diagram of an example of ballast water process equipment.

FIG. 11 is a flowchart of an example of a process procedure for water supply process of ballast water.

FIG. 12 is a flowchart of an example of a process procedure for water discharge process of the ballast water.

FIG. 13 is a diagram of an example of boiler equipment.

FIG. 14 is a flowchart of an example of a process procedure for boiler equipment.

FIG. 15 is a flowchart of an example of a process procedure for a replenishment process for circulation water.

FIG. 16 is a flowchart of an example of a process procedure for water desalination process.

FIG. 17 is a flowchart of an example of a process procedure for an equipment data transmission process.

FIG. 18 is a flowchart of an example of a process procedure for a production and output process for management/process data.

FIG. 19 is a diagram of a modification example of the management center.

FIG. 20 is a diagram of a modification example of a water supply and discharge line.

FIG. 21 is a diagram of an example of a ship selection screen.

FIG. 22 is a diagram of an example of a water process status screen.

FIG. 23 is a diagram of an example of a trend display screen.

FIG. 24 is a diagram of an example of an integral display screen.

FIG. 25 is a diagram of an example of a management data display screen.

FIG. 26 is a diagram of an example of a drug information display screen.

FIG. 27 is a diagram of an example of an overview reporting document.

FIG. 28 is a diagram of an example of a detail reporting document.

FIG. 29 is a diagram of another example of a reporting document.

FIG. 30 is a flowchart of an example of a process procedure for an output process for management/process data for a control apparatus.

FIG. 31 is a flowchart of an example of a process procedure for an output process for management/process data for a management server.

FIG. 32 is a flowchart of an example of a process procedure for an arrangement process for drugs.

### MODES FOR CARRYING OUT THE INVENTION

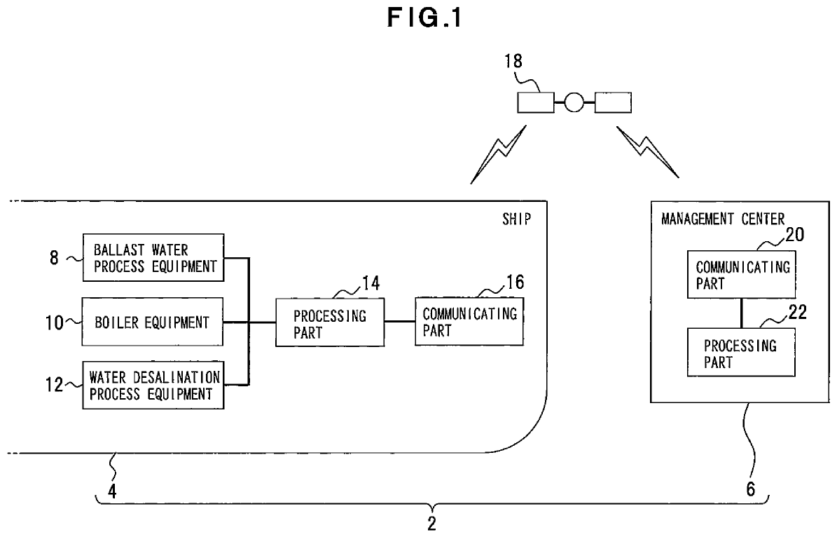

According to an equipment management system 2 for a ship of an embodiment, as depicted in FIG. 1, the equipment management system 2 for the ship includes a ship 4 and a management center 6. The ship 4 has a processing part 14 and a communicating part 16 loaded thereon together with plural pieces of equipment such as ballast water process equipment 8, boiler equipment 10, and water desalination process equipment 12. The ship 4 to be an example sails oceans in various regions and represents one of a myriad of ships. The ballast water process equipment 8 includes a function of executing disinfection of ballast water loaded on the ship 4, and the like and analysis functions such as a total residual oxidants (TRO) meter and a water quality analyzer. The boiler equipment 10 produces steam, high temperature water, warm water, and the like that are used in the ship 4, and includes a valve opening and closing function, analysis functions such as a water quality analyzer, and the like. The water desalination process equipment 12 includes a function of producing drinking water, cooling water, and the like provided for the use in the ship 4 using, for example, seawater as raw water, a valve opening and closing function, analysis functions such as a water quality analyzer, and the like. The ballast water process equipment 8, boiler equipment 10, and water desalination process equipment 12 are an example of pieces of equipment that each needing water process such as disinfection and filtering, and each of these pieces of equipment has a computer loaded thereon that executes information processing and control.

The processing part 14 is connected by a wire or by radio to the ballast water process equipment 8, boiler equipment 10, and water desalination process equipment 12, is in charge of information processing necessary for equipment management of the ship 4, and includes, for example, a computer. The processes executed by the processing part 14 includes a process of producing notification information. The notification information includes one piece of state information or combination information of two or more pieces of state information selected from pieces of state information acquired from at least the ballast water process equipment 8, boiler equipment 10, and water desalination process equipment 12. The communicating part 16 transmits by radio the notification information produced by the processing part 14.

The notification information sent out from the communicating part 16 reaches the management center 6 through a public radio communication satellite 18 that is an example of a public radio communicating apparatus. The management center 6 includes a communicating part 20 and a processing part 22. The communicating part 20 is timely connected to the communicating part 16 of the ship 4 through the public radio communication. The function of the communicating part 20 acquires the notification information from the ship 4 by radio. As above, the notification information includes one piece of state information, or two or more pieces of state information selected from the pieces of state information acquired from at least the ballast water process equipment 8, boiler equipment 10, and water desalination process equipment 12 that are loaded on the ship 4.

The processing part 22 is an example of information processing equipment such as a computer system installed in the management center 6. The processing part 22 extracts the state information of the ballast water process equipment 8, the boiler equipment 10, or the water desalination process equipment 12 from the notification information acquired from the ship 4, produces management information including at least drug information needed by the ballast water process equipment 8, the boiler equipment 10, or the water desalination process equipment 12, and transmits the management information from the communicating part 20 toward at least the ship 4 that transmits the notification information.

The ship 4 and the management center 6 constitute the equipment management system 2 in this embodiment while the equipment management system 2 may be constituted by the processing part 14 and the communicating part 16 of the ship 4, and the management center 6.

### <Process Sequence for Ship 4>

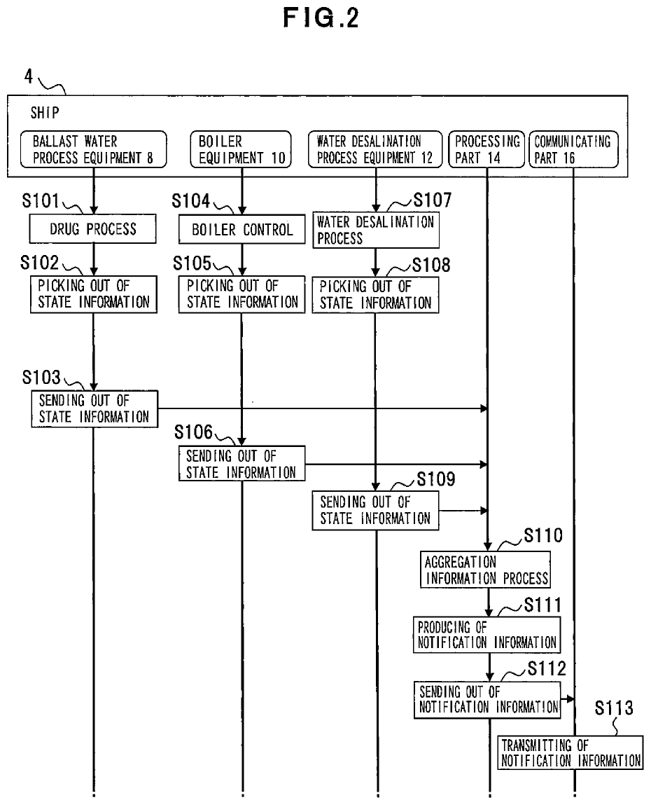

According to the equipment management system 2 for the ship, processes depicted in FIG. 2 are executed by the processing part 14 of the ship 4. These processes are an example of functions executed with a program or a management method. In FIG. 2, “step S” represents a processing stage.

The ballast water process equipment 8 executes a drug process (step S101), executes picking out of the state information such as the effects of the drugs and the water quality of the ballast water (step S102), and sends out the state information to the processing part 14 (step S103).

The boiler equipment 10 executes boiler control (step S104), executes picking out of the state information such as the boiler control and the water quality (step S105), and sends out the state information to the processing part 14 (step S106).

The water desalination process equipment 12 executes a water desalination process (step S107), executes picking out of the state information such as the control state of the water desalination process, the water quality of the produced water, and the like (step S108), and sends out the state information to the processing part 14 (step S109).

The processing part 14 executes information processing of aggregating the provided pieces of state information (step S110). The notification information is produced from the pieces of state information based on the aggregation information process (step S111). The notification information includes one piece of state information, or two or more pieces of state information and is sent out from the processing part 14 to the communicating part 16 (step S112).

The communicating part 16 is controlled by the processing part 14, and transmits the notification information to the management center 6 regularly or in response to instruction information from the management center 6 (step S113).

### <Process Sequences for Management Center 6 and Ship 4>

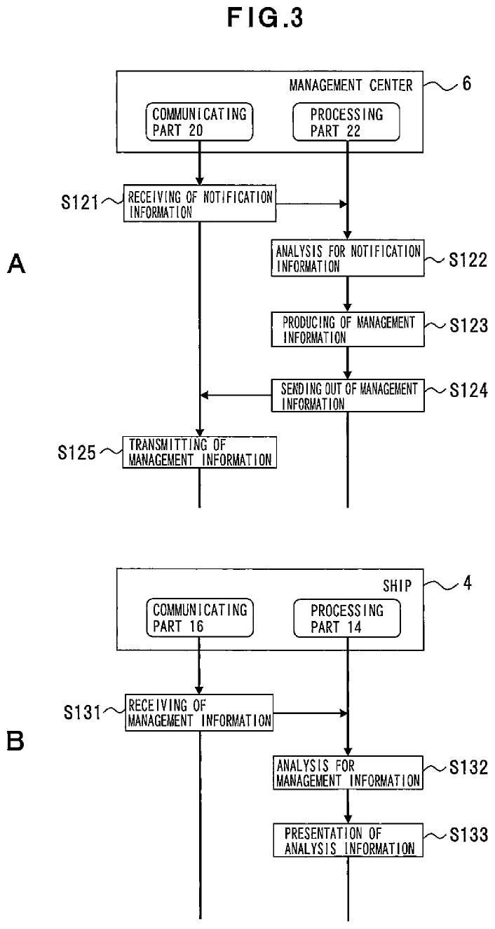

FIG. 3A depicts a process sequence for the management center 6 and FIG. 3B depicts a process sequence for the ship 4. The processes are an example of the functions executed with the program or the management method. In FIG. 3, “step S” represents a processing stage.

In the management center 6, as depicted in FIG. 3A, when the communicating part 20 receives the notification information from the ship 4 (step S121), the notification information is provided to the processing part 22. The processing part 22 executes an analysis for the notification information (step S122), produces the management information as the analysis result (step S123), and sends out the management information to the communicating part 20 (step S124). The communicating part 20 transmits the management information to the ship 4, that is, the ship 4 that transmits the notification information (step S125).

In the ship 4, as depicted in FIG. 3B, when the communicating part 16 receives the management information from the management center 6 (S131), the received management information is provided from the communicating part 16 to the processing part 14. The processing part 14 executes an analysis for the management information (step S132) and executes presentation of the analysis information as the analysis result (step S133). The analysis information is provided to the corresponding ballast water process equipment 8, the corresponding boiler equipment 10, and the corresponding water desalination process equipment 12, or is displayed on a displaying means controlled by the processing part 14. Any measure necessary for each of the pieces of equipment 8, 10, and 12 such as the drug for the ballast water only has to be taken based on the management information.

### Effect of Embodiment

(1) According to the equipment management system 2, the pieces of state information on the ballast water process equipment 8, the boiler equipment 10, and the water desalination process equipment 12 can be collectively managed and the notification information to be the collective information can timely be received by the management center 6. The pieces of state information on the ballast water process equipment 8, the boiler equipment 10, and the water desalination process equipment 12 can each independently be notified of separately from each other, and speeding up is enabled for the steps from acquisition of the necessary information to placing an order for the drugs and the like, the necessary operations and control, and the like.

(2) The pieces of notification information from the plural ships 4 interspersed in various regions can be managed for each of the ships, and simplification of the management and speeding up of the processing can be facilitated.

(3) The necessary measures such as procurement of the drugs and necessary engineers can be taken for an anchorage site without waiting for the arrival of the ship 4 thereto.

### Example 1

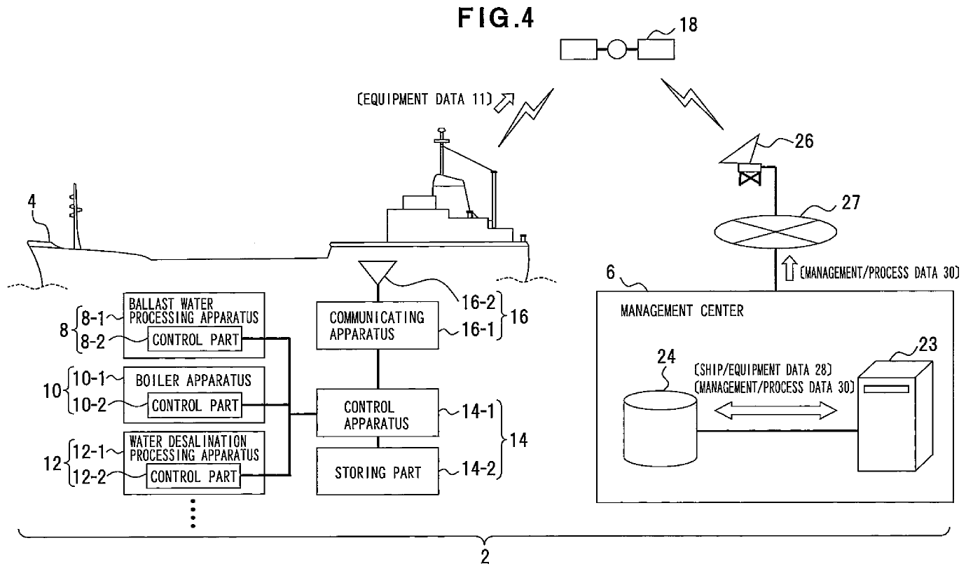

Example 1 will be described with reference to FIG. 4. FIG. 4 is a diagram of an example of the equipment management system for the ship according to Example 1. Portions same as or corresponding to those in FIG. 1 are given the same reference numerals. The equipment management system 2 includes the ship 4 and the management center 6. The ship 4 has a control apparatus 14-1, a storing part 14-2, a communicating apparatus 16-1, and an antenna 16-2 loaded thereon together with plural pieces of water process equipment relating to water processes such as a ballast water processing apparatus 8-1, a boiler apparatus 10-1, and a water desalination processing apparatus 12-1. The management center 6 includes a management server 23 and a database 24. The ship 4 and the management center 6 are connected to each other by radio.

The ballast water processing apparatus 8-1 is an example of the ballast water process equipment 8. The ballast water processing apparatus 8-1 includes a control part 8-2 that outputs the state information on the control and the process for the ballast water process. The boiler apparatus 10-1 is an example of the boiler equipment 10. The boiler apparatus 10-1 includes a control part 10-2 that outputs the state information indicating the control state of the boiler control, the water quality and the like. The water desalination processing apparatus 12-1 is an example of the water desalination process equipment 12. The water desalination processing apparatus 12-1 includes a control part 12-2 that outputs the state information on the water desalination control and the control for the water quality and the like.

The control apparatus 14-1 and the storing part 14-2 are an example of the processing part 14. The control apparatus 14-1 executes the programs stored in the storing part 14-2 and facilitates the cooperation of the control parts 8-2, 10-2, and 12-2 among each other. The control apparatus 14-1 receives the provision of the plural pieces of state information output from the control parts 8-2, 10-2, and 12-2, and produces the notification information that includes one piece, or two or more pieces of state information.

The notification information produced by the control apparatus 14-1 is transmitted from the antenna 16-2 toward the management center 6 in accordance with the control by the control apparatus 14-1 through the communicating apparatus 16-1 and the antenna 16-2 that are an example of the communicating part 16. The antenna 16-2 is connected to the communicating apparatus 16-1.

The control apparatus 14-1 is connected by a wire or radio to the ballast water processing apparatus 8-1, the boiler apparatus 10-1, and the water desalination processing apparatus 12-1, and transmits pieces of equipment data 11 acquired from at least the ballast water processing apparatus 8-1, the boiler apparatus 10-1, and the water desalination processing apparatus 12-1 to the management center 6 through the communicating apparatus 16-1 and the public radio communication satellite 18. The equipment data 11 is an example of the state information and the notification information described in the embodiment.

The control apparatus 14-1 is provided with the storing part 14-2. The control apparatus 14-1 includes a computer that has an information processing function and is, for example, a personal computer (PC), a tablet PC, or a smartphone. The control apparatus 14-1 stores in the storing part 14-2 the pieces of equipment data 11 received from the ballast water processing apparatus 8-1, the boiler apparatus 10-1, and the water desalination processing apparatus 12-1, attaches an equipment index to identify the equipment to each of the pieces of equipment data 11, and outputs the pieces of equipment data 11 each having the equipment index attached thereto, to the communicating apparatus 16-1.

In the control and the information processing by the control apparatus 14-1, for example, the pieces of equipment data 11 are combined with each other for each type of water for one piece of equipment and the equipment index is attached to the pieces of equipment data 11 for each type of water for one piece of equipment. When no other piece of equipment data 11 to be combined with is present, or the like, the equipment index may be attached to one piece of equipment data 11. The equipment data 11 to which the equipment data is attached in this manner may be output alone, or the plural pieces of equipment data 11 may be combined with each other and may collectively be output regularly or irregularly. The pieces of equipment data 11 are aggregated by this data combining process.

The equipment index only has to be attached based on, for example, the equipment information provided from the management server 23. For example, a file name in the equipment information set for each piece of equipment and each type of water may be used as the equipment index. In this case, the control apparatus 14-1 only has to attach a file name that corresponds to each piece of equipment and each type of water, to the equipment data 11.

The control apparatus 14-1 further stores, displays, or prints management/process data 30 received from the management center 6. The management/process data 30 is an example of the management information described in the embodiment.

The ballast water processing apparatus 8-1 is an apparatus that processes the ballast water used for stabilization of the ship. The ballast water is injected into, for example, a tank at the ship bottom. The ballast water processing apparatus 8-1 processes the ballast water in accordance with the control by the control part 8-2 to, for example, satisfy the “Ballast Water Management Standards”. This “Ballast Water Management Standards” are stipulated in Section D of “Regulations for the Control and Management of Ships' Ballast Water and Sediments” that is Annex of the “International Convention for the Control and Management of Ships' Ballast Water and Sediments, 2004” adopted in February 2004 (hereinafter, referred to as “ballast water management convention”). When the ballast water is injected, the ballast water processing apparatus 8-1 injects a disinfectant of a chlorinated active substance such as sodium hypochlorite or calcium hypochlorite into the ballast water and thereby annihilates the microorganisms and the like therein. Before the ballast water is discharged, the ballast water processing apparatus 8-1 neutralizes the ballast water using a neutralizer. The ballast water processing apparatus 8-1 manages the equipment data 11. The equipment data 11 is pieces of information on the use amounts and the remaining amounts of each of the drugs such as the disinfectant, the neutralizer, and the like in addition to operation information on the ballast water processing apparatus 8-1, pieces of information on the water quality of the ballast water, the injection amount of the ballast water, the discharge amount of the ballast water, and the remaining amount of the ballast water.

The boiler apparatus 10-1 produces steam necessary for the sailing of the ship 4 or steam necessary in the ship. The boiler apparatus 10-1 is, for example, a main boiler that produces steam to drive the steam turbine of the main engine, an exhaust gas boiler that produces steam using the heat of the exhaust gas, an oil burning boiler that generates steam using combustion heat of oil, or a composite boiler that generates steam by causing the exhaust gas to pass through an oil burning boiler. Process water BW is used to produce the steam by the boiler apparatus 10-1. The process water BW is concentrated by being used for a long time. Slime and scales are generated inside the pipes by the impurities included in the process water BW. In the boiler apparatus 10-1, the water quality of the process water BW is therefore measured and analyzed regularly or irregularly to be managed. In the boiler apparatus 10-1, the equipment data 11 is managed. The equipment data 11 managed in the boiler apparatus 10-1 is information on the remaining amount and the like of each of the drugs used in the boiler apparatus 10-1 such as a scale preventive, an anticorrosive, a sludge dispersant, a disinfectant, and a slime inhibitor, in addition to operation information on the boiler apparatus 10-1 and information on the water quality of the process water BW used in the boiler apparatus 10-1, and the like.

The water desalination processing apparatus 12-1 produces clean water from the used water produced in the ship 4, freshwater such as rain water, or seawater. This clean water is produced by, for example, ion removal using an electric double layer, a desalination process, electro-dialysis, filtering using a reverse osmosis membrane, or distillation. The produced clean water is used for, for example, the process water BW of the boiler apparatus 10-1, the cooling water of cooling equipment, domestic-use water, and general service water. The water quality of the clean water produced by the water desalination processing apparatus 12-1 is measured and analyzed regularly or irregularly to be managed. In the water desalination processing apparatus 12-1, the equipment data 11 is managed. The equipment data 11 managed in the water desalination processing apparatus 12-1 is information on the remaining amount and the like of each of the drugs used in the water desalination processing apparatus 12-1 such as a scale preventive, an anticorrosive, a mineral agent, and a cleaning agent for pipes and tanks, in addition to operation information on the water desalination processing apparatus 12-1 and information on the water quality of the clean water produced by the water desalination processing apparatus 12-1, and the like.

The communicating apparatus 16-1 is an example of a radio communicating apparatus that communicates with the land through the public radio communication satellite 18. The communicating apparatus 16-1 is provided with the antenna 16-2, and the communicating apparatus 16-1 is connected to the control apparatus 14-1. The communicating apparatus 16-1 modulates the equipment data 11 received from the control apparatus 14-1 into a radio signal and outputs the radio signal from the antenna 16-2. The communicating apparatus 16-1 demodulates the radio signal received by the antenna 16-2 into data and outputs the data to the control apparatus 14-1.

The public radio communication satellite 18 is a communication satellite such as, for example, an Inmarsat satellite. The public radio communication satellite 18 connects the communicating apparatus 16-1 and communication equipment 26 installed on the land to each other by radio. The communicating apparatus 16-1 and the communication equipment 26 communicate with each other by radio through the public radio communication satellite 18. The radio communication line by the public radio communication satellite 18 and the communication equipment 26 is provided by, for example, a communication company.

The communication equipment 26 is connected to the management center 6 through a communication network 27. The communication network 27 is an example of a communication line that transmits the data, only has to be able to transmit the data, and is, for example, the Internet or an intranet.

The management server 23 is an example of the managing apparatus that manages the pieces of water process equipment such as the ballast water processing apparatus 8-1, the boiler apparatus 10-1, and the water desalination processing apparatus 12-1 based on the pieces of equipment data 11 received from the communicating apparatus 16-1. The management server 23 produces ship/equipment data (ship and equipment data) 28 by correlating the pieces of equipment data 11 received from the communicating apparatus 16-1 with the pieces of equipment information stored in the database 24, and stores the ship/equipment data 28 in the database 24. The database 24 therefore has the pieces of ship/equipment data 28 accumulated therein.

The management server 23 produces the management/process data 30 relating to the pieces of water process equipment for each ship using the ship/equipment data 28, stores the management/process data 30 in, for example, the database 24, and provides the management/process data 30 to the customers engaged in the ship through the communication network 27. The customers engaged in the ship are, for example, the ship 4 that transmits the pieces of equipment data 11, the ship owner owning the ship 4 (such as, for example, the company owning the ship), and the ship manager managing the ship 4 (such as, for example, the ship managing company). The management server 23 repeats the storing of the management/process data 30 into the database 24 and provision thereof to the customers. The database 24 therefore has the pieces of management/process data 30 accumulated therein, and collective management of and collective provision to the customers of the pieces of equipment data 11 are facilitated.

The management/process data 30 is data relating to, for example, the analysis result of the equipment data 11, the diagnosis results of the pieces of water process equipment, or a trend graph that shows the tendency of the pieces of equipment data 11, and includes at least either the process data or the management data of each of the pieces of water process equipment. The process data is the data indicating the process state of each of the pieces of water process equipment and includes, for example, the process amount by the water process equipment, the injection amount of the drug into the water process equipment, the result of the measurement or the analysis of the water used in the water process equipment or the water produced by the water process equipment. The management data is the data necessary for the management of the pieces of water process equipment and includes, for example, drug information such as the retention amounts, the temperatures or the concentrations of the drugs used in the pieces of water process equipment, the alarm history of the pieces of water process equipment, the power consumption of each of the pieces of water process equipment, the information on the injection place of the ballast water, and the information on the discharge place of the ballast water. These pieces of data are an example and the present invention is not limited by the described pieces of data. For the data corresponding to both of the process data and the management data, this data only has to be handled as either the process data or the management data.

The management/process data 30 only has to be produced using, for example, a data analyzing function, a data diagnosing function, and a data processing function included in the management server 23. The data analyzing function of the management server 23 analyzes the ship/equipment data 28 stored in the database 24, produces the average value of the pieces of equipment data 11 included in, for example, the ship/equipment data 28 (averaging of the pieces of equipment data 11), calculates the ascending velocity and the descending velocity of the pieces of equipment data 11, and thereby diagnoses the trend (the tendency) of the pieces of equipment data 11 (trend diagnosis). The data analyzing function converts the pieces of equipment data 11 into index values through the averaging of the pieces of equipment data 11 and the trend diagnosis, and analyzes the pieces of equipment data 11 converted into the index values. The data diagnosing function of the management server 23 diagnoses the analyzed data, compares, for example, the index values acquired by the data analyzing function with threshold value used for determination of equipment abnormality, and detects and diagnoses any abnormality of any one of the pieces of water process equipment. The data processing function of the management server 23 processes the equipment data 11 such as processing the data into a format suitable for displaying or printing, and producing a trend graph based on, for example, the same type of pieces of chronological data acquired at different times of day.

The database 24 accumulates therein and assembles the pieces of data relating to the pieces of water process equipment such as the ballast water processing apparatus 8-1, the boiler apparatus 10-1, and the water desalination processing apparatus 12-1. The database 24 is formed by a storage apparatus such as, for example, a magnetic storage apparatus, an optical disc, or a semiconductor memory. The database 24 may be a network service such as, for example, cloud computing and may be installed outside the management center 6. The database 24 includes an operation database and an equipment information database. The operation database stores therein the ship/equipment data 28 and the management/process data 30. The equipment information database stores therein the equipment information that includes the installation ship, the equipment name, the equipment type, and the equipment specification of each of the pieces of water process equipment. The equipment information database may further store therein the sailing schedule of the ship, and the equipment information such as the maintenance history of the ship equipment. The equipment information database only has to be set when plural pieces of water process equipment to be managed are present. The database 24 is connected to the management server 23 by a wire or by radio.

### <Equipment Information>

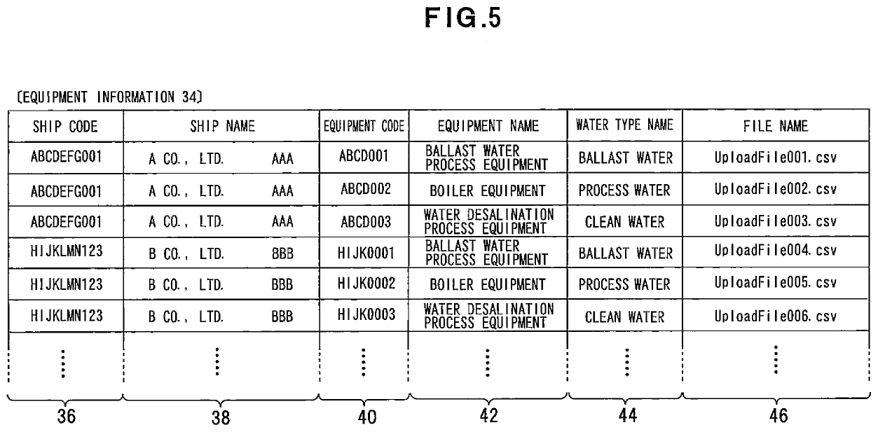

FIG. 5 depicts an example of the equipment information stored in the equipment information database.

The equipment information 34 includes, for example, a ship code 36, ship name information 38, an equipment code 40, equipment name information 42, water type name information 44, and file name information 46. The ship code 36 and the ship name information 38 each indicate the ship retained by the customers. The ship code 36 is, for example, a 10-digit character string “ABCDEFG001” and includes alphabets, digits, or a combination of alphabets and digits. The ship name information 38 corresponds to the ship code 36 and includes, for example, the company name “A Co., Ltd.” and the ship name “AAA”.

The equipment code 40 and the equipment name information 42 represent each of the pieces of water process equipment installed in the ship 4. The equipment code 40 is, for example, a seven-digit character string “ABCD001” and includes alphabets, digits, or a combination of alphabets and digits. The equipment name information 42 corresponds to the equipment code 40 and includes the names of the pieces of equipment such as “ballast water process equipment”, “boiler equipment”, and “water desalination process equipment”. The water type name information 44 indicates the type of water processed by each of the pieces of equipment and includes, for example, process water name such as “ballast water”, “process water”, and “clean water”. The data of the process water listed as the process water name is collected by the ship and is analyzed and managed by the land.

The file name information 46 indicates the data transmission specification used when the communicating apparatus 16-1 transmits the equipment data 11 to the management server 23. The management server 23 compares the file name attached to the equipment data 11 with the file name information 46 of the equipment information 34, identifies the equipment relating to the equipment data 11, and correlates the equipment data 11 with the equipment information 34. The file name information 46 is, for example, a transmission CSV file name and is “UploadFile001.csv” as an example.

### <Ship/Equipment Data 28>

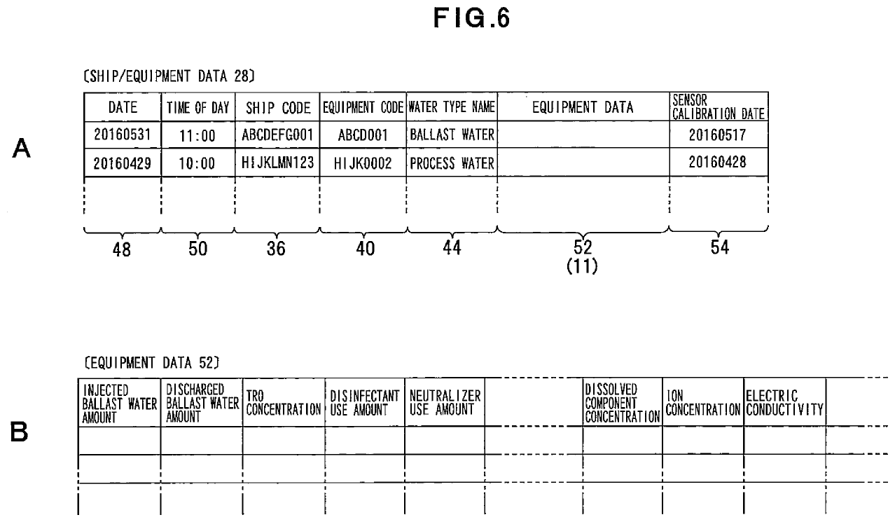

FIG. 6 each depict an example of the ship/equipment data 28.

As depicted in FIG. 6A, the ship/equipment data 28 includes, for example, date information 48, time of day information 50, the ship code 36, the equipment code 40, the water type name information 44, equipment data 52, and sensor calibration day information 54. The equipment data 52 is the data that forms a database using the pieces of equipment data 11 received from the ship 4. The ship code 36 is the information to identify the ship 4 that transmits the equipment data 52, and the equipment code 40 and the water type name information 44 are the pieces of information to identify the equipment and the water type relating to the equipment data 52.

The date information 48 indicates the date of the day for the equipment data 52 to be collected or analyzed, and the time of day information 50 indicates the time of day for the equipment data 52 to be collected or analyzed. The sensor calibration day information 54 indicates the calibration day for the sensor used in the acquisition or the analysis of the equipment data 52.

As depicted in FIG. 6B, the equipment data 52 is, for example, the injected water amount of the ballast water, the discharged water amount of the ballast water, the TRO concentration of the ballast water, the use amount of the disinfectant, and the use amount of the neutralizer. The TRO is the total residual oxides, and is an example of the disinfection index for the microorganisms and the like in the ballast water and the neutralization index of the ballast water. The equipment data 52 is, for example, the dissolved component concentration, the ion concentration, and the electric conductivity of the process water. The ship/equipment data 28 is an example and may further include the operation information indicating the start of starting up or the stoppage of the equipment and information such as, the water amount of the water processed, produced, or used by the equipment, water pressure information, or the input amounts of the drugs used in the process of water processed, produced, or used in the pieces of equipment, as the pieces of information each indicating the operation state of the equipment.

### <Management/Process Data 30>

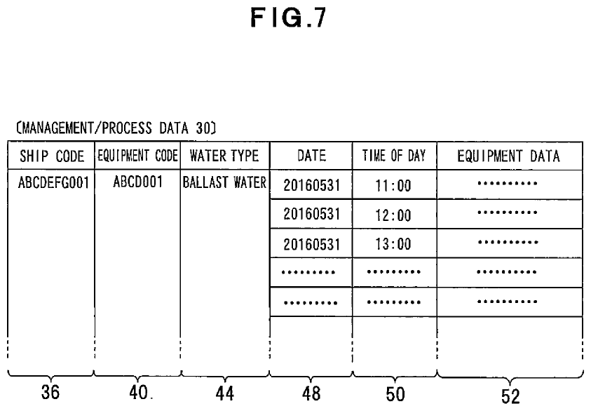

FIG. 7 depicts an example of the management/process data. The management/process data 30 includes, for example, the ship code 36, the equipment code 40, the water type name information 44, the date information 48, the time of day information 50, and the equipment data 52. In the management/process data 30, the pieces of equipment data 52 relating to the water of each of the pieces of equipment of the specific ship 4 are gathered. The management/process data 30 stores therein in the chronological order pieces of specific equipment data 52 whose pieces of time of day information 50 differ from each other, and indicates the tendency of the pieces of equipment data 11 relating to the elapse of time. The management/process data 30 is usable for producing the trend graph. The management/process data 30 is not limited to the table-formed one as depicted in FIG. 7 and may be stored in the database in a form to show the analysis result of the equipment data 11 or the diagnosis result of the equipment.

### <Processing Part 14>

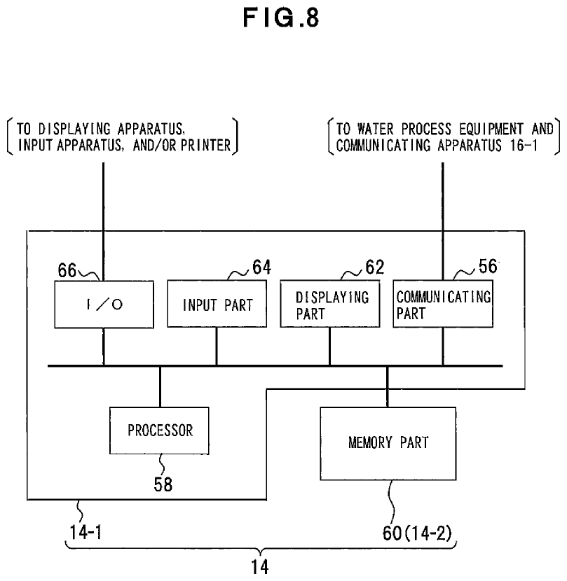

FIG. 8 depicts an example of the hardware configuration of the processing part 14. The control apparatus 14-1 of the processing part 14 includes a communicating part 56, a processor 58, a displaying part 62, an input part 64, and an input-output (I/O) 66.

The communicating part 56 is connected to the pieces of water process equipment and the communicating apparatus 16-1, and communicates data with the pieces of water process equipment and the communicating apparatus 16-1. The communicating part 56 is connected to the pieces of water process equipment such as the ballast water processing apparatus 8-1, the boiler apparatus 10-1, and the water desalination processing apparatus 12-1, and the communicating apparatus 16-1 by a wire through a serial communication cable such as, for example, RS-232C or a universal serial bus (USB), or a local area network (LAN) cable. The communicating part 56 may be connected to the pieces of water process equipment and the communicating apparatus 16-1 by radio through, for example, a wireless LAN such as wireless fidelity (Wi-Fi).

The processor 58 is an example of an information processing part that processes information and is, for example a central processing unit (CPU). The processor 58 executes an operating system (OS) and equipment management program stored in a memory part 60 and executes various types of information processing. The information processing executed by the processor 58 includes communication control at the communicating part 56, displaying control at the displaying part 62, input control at the input part 64, and input and output control at the I/O 66.

The memory part 60 is an example of the storage apparatus that stores therein the programs such as the OS and the equipment management program to be executed by the processor 58, and that also stores therein the pieces of equipment data 11 received from the pieces of water process equipment, the equipment information 34 and the management/process data 30 received from the management server 23. The memory part 60 executes storing and reading of the various types of information in accordance with the control by the processor 58. The memory part 60 includes one or plural storage element (s) such as a read-only memory (ROM), a random-access memory (RAM), an electrically erasable programmable read-only memory (EEPROM), a NAND-type flash memory, and an NOR-type flash memory. A hard disc apparatus or a semiconductor storage device may be used as the storage element.

The displaying part 62 includes a display such as, for example, a liquid crystal display, a light emitting diode (LED) display, or an organic EL display, and displays thereon the analysis result of the pieces of equipment data 11, the diagnosis results of the pieces of water process equipment, the trend graph, or the like based on the management/process data 30 acquired from the management server 23. The input part 64 is, for example, a keyboard, a mouse, a touch panel, or hardware keys, accepts an operation by a user, produces operation information based on this operation, and outputs the operation information to the processor 58. The input part 64 accepts, for example, inputs of an identification (ID) of the user who uses the control apparatus 14-1 and a password to unlock the lock of the control apparatus 14-1. The I/O 66 is connected to a peripheral apparatus and communicates data with the peripheral apparatus. The peripheral apparatus is, for example, an external displaying apparatus, an external input apparatus, or a printer.

### <Management Server 23>

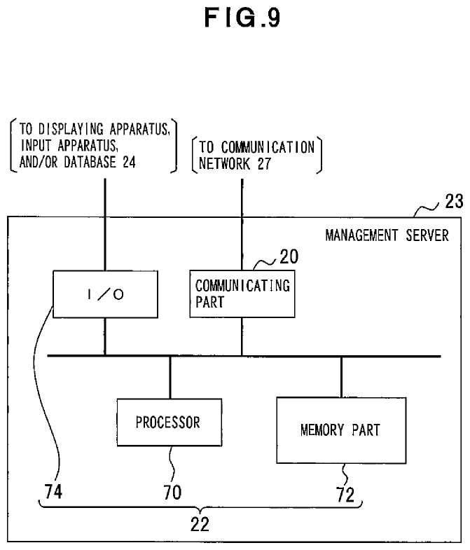

FIG. 9 depicts an example of the hardware configuration of the management server. The management server 23 includes the processing part 22 that includes the communicating part 20, a processor 70, a memory part 72, and an I/O 74. Portions same as those in FIG. 1 are given the same reference numerals.

The communicating part 20 is connected to the communication network 27 and communicates data with the communication network 27. The communicating part 20 is connected to the communication network 27 by a wire using, for example, a LAN cable. The communicating part 20 may be connected to the communication network 27 by radio through, for example, a radio LAN such as Wi-Fi.

The processor 70 is an example of the information processing part that processes information and is, for example, a central processing unit. The processor 70 executes an OS and equipment management program stored in the memory part 72, executes the data analyzing function, the data diagnosing function, and the data processing function, causes the database 24 to read and write data, and provides the management/process data 30 to the database 24.

The memory part 72 is an example of a storage apparatus that stores therein the programs such as the OS and the equipment management program to be executed by the processor 70. The memory part 72 may be used for storing therein the ship/equipment data 28, the management/process data 30, or the equipment information 34. The memory part 72 executes storing or reading of the various types of information in accordance with the control by the processor 70. The memory part 72 includes one or plural storage element(s) such as a ROM, a RAM, an EEPROM, a NAND-type flash memory, and a NOR-type flash memory. A hard disc apparatus or a semiconductor storage device may be used as this storage element.

The I/O 74 is connected to the displaying apparatus, the input apparatus, and the database 24 and is used for inputting and outputting of image data, inputting of operation information produced by an operation on an input apparatus, and transmission and reception of data with the database 24.

### <Ballast Water Process Equipment 8>

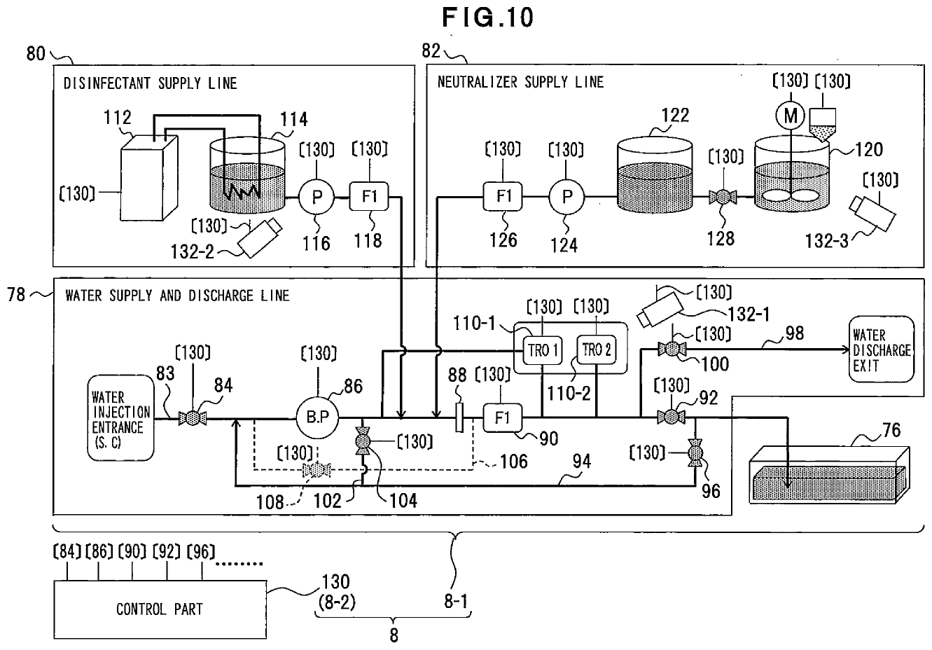

FIG. 10 depicts an example of the ballast water process equipment 8 that includes the ballast water processing apparatus 8-1 and its control part 130.

The ballast water processing apparatus 8-1 includes a ballast tank 76, a water supply and discharge line 78 connected to the ballast tank 76, a disinfectant supply line 80 connected to the water supply and discharge line 78, and a neutralizer supply line 82.

The water supply and discharge line 78 includes a main line 83 and the main line 83 includes a ballast water injection valve 84, a ballast pump (B.P) 86, a mixer 88, a ballast flowmeter (F1) 90, and a ballast tank entrance valve 92. The ballast water injection valve 84 is installed downstream an water injection entrance (S.C) that takes in the ballast water from the sea, and causes the ballast water supplied from the water injection entrance to pass therethrough or closes in accordance with the opening or closing of the valve. The ballast pump 86 is installed on the downstream side of the ballast water injection valve 84 and causes the ballast water to flow to the mixer 88 by being driven. The mixer 88 is installed downstream the ballast pump 86 and mixes the disinfectant or the neutralizer injected between the ballast pump 86 and the mixer 88, with the ballast water. The ballast flowmeter 90 is installed downstream the mixer 88 and measures the flow amount of the ballast water that includes the disinfectant or the neutralizer. The ballast tank entrance valve 92 is installed downstream the ballast flowmeter 90 and causes the ballast water to pass therethrough or closes by opening or closing of the valve. The ballast water passing through the ballast tank entrance valve 92 is poured into the ballast tank 76.

The water supply and discharge line 78 further includes a branch line 94 disposed in parallel to the main line 83, and a water discharge line 98. The branch line 94 includes a ballast tank exit valve 96 and the water discharge line 98 includes a ballast water discharge valve 100. One end of the branch line 94 is connected to a point between the ballast tank entrance valve 92 and the ballast tank 76, and the other end thereof is connected to a point between the ballast water injection valve 84 and the ballast pump 86. The water discharge line 98 is connected to the main line 83 at a point between the ballast flowmeter 90 and the ballast tank entrance valve 92. The branch line 94 and the water discharge line 98 are used to discharge the ballast water.

The ballast tank exit valve 96 causes the ballast water in the branch line 94 to pass therethrough or closes by the opening or closing of the valve. The ballast water discharge valve 100 causes the ballast water in the water discharge line 98 to pass therethrough or closes by the opening or closing of the valve. When the ballast water injection valve 84 and the ballast tank entrance valve 92 are closed and the ballast tank exit valve 96 and the ballast water discharge valve 100 are opened, a water discharge path for the ballast water is formed. When the ballast pump 86 is caused to operate, the ballast water in the ballast tank 76 flows through the branch line 94, the ballast pump 86, the mixer 88, and the ballast flowmeter 90 in this order, passes through the water discharge line 98, and is discharged from the water discharge exit to the sea.

The water supply and discharge line 78 includes a gravity water injection and discharge line 102 and a gravity water injection and discharge valve 104. The gravity water injection and discharge line 102 connects the branch line 94 to the main line 83 between the ballast pump 86 and the mixer 88. When the gravity water injection and discharge valve 104 disposed in the gravity water injection and discharge line 102 is opened, a bypass route bypassing the ballast pump 86 is formed, and supply and discharge of the ballast water by the gravity are thereby enabled.

The water supply and discharge line 78 further includes a bypass line 106 and the bypass line 106 may include a bypass valve 108. The bypass line 106 and the bypass valve 108 form a bypass route for the line from the ballast pump 86 to the mixer 88.

The water supply and discharge line 78 further includes a first TRO meter (TRO 1) 110-1 and a second TRO meter (TRO 2) 110-2 that each measure the TRO of the ballast water. The TRO meter 110-1 measures the TRO at a first TRO measurement point set between the connection point of the gravity water injection and discharge line 102 and the mixer 88. At the first TRO measurement point, the TRO meter 110-1 measures the high concentration TRO before the injection of the neutralizer. The measurement result acquired at the first TRO measurement point together with the flow amount by the ballast flowmeter 90 is used for determination of the injection amount of the neutralizer during the ballast water discharge. The TRO meter 110-1 further measures the TRO at a second TRO measurement point set between the ballast flowmeter 90 and the ballast tank entrance valve 92. At the second TRO measurement point, the TRO meter 110-1 measures the high concentration TRO after the injection of the disinfectant. The measurement result acquired at the second TRO measurement point is used for determination as to whether the concentration of the TRO after the injection of the disinfectant is proper and feedback control of the injection amount of the disinfectant. The TRO meter 110-2 measures the TRO at a third TRO measurement point between the ballast flowmeter 90 and the ballast tank entrance valve 92. At the third TRO measurement point, the TRO meter 110-2 measures the TRO after the injection of the neutralizer. The measurement result acquired at the third TRO measurement point is used for determination as to whether the neutralization of the ballast water by the neutralizer is proper. The TRO meters 110-1 and 110-2 are each a measuring device using, for example, diethyl-P-phenylenediamine (DPD) absorption photometry. The TRO meter using the DPD absorption photometry measures the residual chlorine concentration from the absorption rate of light of the ballast water having a DPD reagent added thereto.

The disinfectant supply line 80 is connected to the main line 83 between the first TRO measurement point and the mixer 88 and injects the disinfectant into the ballast water. The disinfectant supply line 80 includes a disinfectant cooling chiller 112, a disinfectant tank 114, a disinfectant injection pump (P) 116, and a disinfectant flowmeter (F1) 118.

The disinfectant cooling chiller 112 is an example of a cooling apparatus that cools down the disinfectant. The disinfectant cooling chiller 112 is connected to the disinfectant tank 114, cools down the disinfectant in the disinfectant tank 114 to a temperature in, for example, a range of 2 to 20° C. and, preferably, a range of 2 to 10° C., and thereby suppresses any degradation of the disinfectant caused by a temperature increase thereof. The disinfectant injection pump 116 injects the disinfectant in the disinfectant tank 114 into the ballast water through the disinfectant flowmeter 118. The disinfectant flowmeter 118 measures the flow amount of the disinfectant injected into the ballast water.

The neutralizer supply line 82 is connected to the main line 83 between the first TRO measurement point and the mixer 88, and injects the neutralizer into the ballast water. The neutralizer supply line 82 includes a neutralizer dissolving tank 120, a neutralizer tank 122, a neutralizer injection pump (P) 124, a neutralizer flowmeter (F1) 126, and a neutralizer supply valve 128. The neutralizer dissolving tank 120 includes a stirring apparatus, produces a neutralizer at a predetermined concentration, and retains the neutralizer for a predetermined time period. For example, sodium sulfite, sodium bisulfite (sodium hydrogen sulfite), sodium thiosulfate, and the like are each usable as the neutralizer. It is especially preferred that sodium thiosulfate be used as the neutralizer. The neutralizer dissolving tank 120 includes an alarming apparatus that, when the raw material of the neutralizer is insufficient, warns for replenishment of the raw material. The neutralizer dissolving tank 120 is connected to the neutralizer tank 122 through the neutralizer supply valve 128. When the neutralizer supply valve 128 is opened, the neutralizer in the neutralizer dissolving tank 120 moves to the neutralizer tank 122. The neutralizer injection pump 124 injects the neutralizer in the neutralizer tank 122 into the ballast water through the neutralizer flowmeter 126. The neutralizer flowmeter 126 measures the flow amount of the neutralizer to be injected into the ballast water.

The ballast water process equipment 8 further includes the control part 130 that controls the ballast water process equipment 8. The control part 130 is an example of the control part 8-2, is connected to the valves, the pumps, the measuring devices, and the disinfectant cooling chiller 112, receives the measurements of the measuring devices, and controls opening and closing of each of the valves, operation and stoppage of each of the pumps, and operation or stoppage of the disinfectant cooling chiller 112. The control part 130 records the pieces of equipment data 11 acquired from the measuring devices, the valves, the pumps, and the disinfectant cooling chiller 112 each with the date and time of the work, and transmits the pieces of equipment data 11 to the control apparatus 14-1.

The ballast water process equipment 8 includes shooting apparatuses 132-1, 132-2, and 132-3 each including, for example, a camera. The shooting apparatus 132-1 is installed in the vicinity of the mixer 88, the ballast flowmeter 90, and the TRO meters 110-1 and 110-2, and shoots the mixer 88, the ballast flowmeter 90, and the TRO meters 110-1 and 110-2. The shooting apparatus 132-2 is installed in the vicinity of the disinfectant supply line 80 and shoots the disinfectant supply line 80. The shooting apparatus 132-3 is installed in the vicinity of the neutralizer supply line 82 and shoots the neutralizer supply line 82. The shooting results of the shooting apparatuses 132-1, 132-2, and 132-3 are distributed from the shooting apparatuses 132-1, 132-2, and 132-3 to the network of the ship 4 through the control part 130 or at first hand or are transmitted to the management server 23 through the communicating apparatus 16-1.

### <Process Procedure for Ballast Water Process Equipment 8>

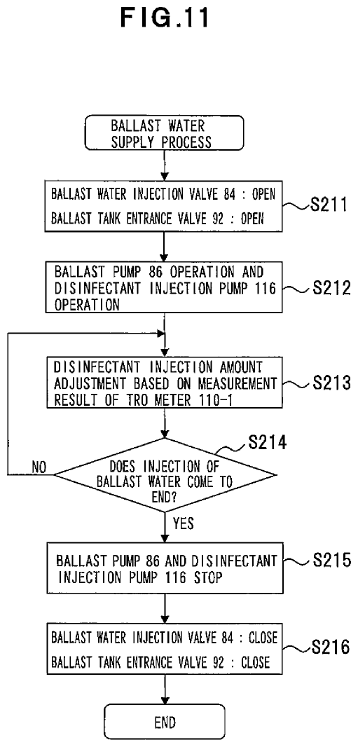

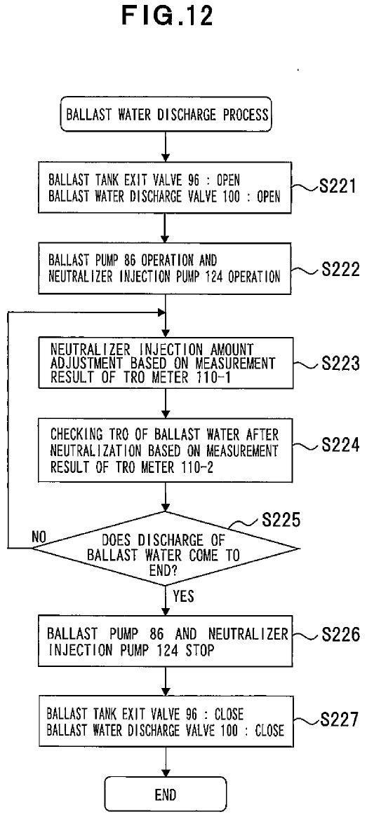

FIG. 11 depicts an example of a process procedure for the water supply of the ballast water and FIG. 12 depicts an example of a process procedure for the water discharge of the ballast water. These processes are executed by the control part 130 of the ballast water process equipment 8. The processes are an example of the functions executed with the program or the management method. In FIG. 11 and FIG. 12, “step S” represents a processing step.

In the water supply process of the ballast water, the control part 130 opens the ballast water injection valve 84 and the ballast tank entrance valve 92 (step S211), operates the ballast pump 86 and the disinfectant injection pump 116 (step S212), and thereby starts the supply of the ballast water and the injection of the disinfectant. The control part 130 adjusts the injection amount of the disinfectant based on the measurement result of the TRO meter 110-1 at the second TRO measurement point (step S213). This adjustment of the injection amount is executed by, for example, adjusting the disinfectant injection pump 116. The concentration of the TRO of the ballast water is adjusted to be, for example, 2 to 30 [mg/L] as Cl₂.

The control part 130 determines whether the injection of the ballast water comes to an end (step S214). When the control part 130 determines that the injection does not yet come to an end (NO of step S214), the control part 130 repeats step S213 and step S214. When the control part 130 determines that the injection comes to an end (YES of step S214), the control part 130 stops the ballast pump 86 and the disinfectant injection pump 116 (step S215) and thereby causes the supply of the ballast water and the injection of the disinfectant to come to an end. The control part 130 closes the ballast water injection valve 84 and the ballast tank entrance valve 92 (step S216), and thereby causes the process of supplying the ballast water to come to an end.

In the water supply process of the ballast water, the control part 130 records the flow amount of the ballast water measured by the ballast flowmeter 90, the flow amount of the disinfectant measured by the disinfectant flowmeter 118, and the concentration of the TRO measured by the TRO meter 110-1 each with the date and time of the work. The amount of the ballast water in the ballast tank 76 and the like are acquired from the flow amount of the ballast water and the remaining amount and the like of the disinfectant in the disinfectant tank 114 is acquired from the flow amount of the disinfectant. The control part 130 transmits these recorded pieces of equipment data 11 to the control apparatus 14-1.

In the water discharge process for the ballast water, the control part 130 opens the ballast tank exit valve 96 and the ballast water discharge valve 100 (step S221), operates the ballast pump 86 and the neutralizer injection pump 124 (step S222), and thereby starts the discharge of the ballast water and injection of the neutralizer. The control part 130 adjusts the injection amount of the neutralizer based on the measurement result of the TRO meter 110-1 at the first TRO measurement point (step S223) and checks the TRO of the ballast water after the neutralization based on the measurement result of the TRO meter 110-2 at the third measurement point (step S224). This adjustment of the injection amount of the neutralizer is executed by, for example, adjusting the neutralizer injection pump 124. The concentration of the TRO of the ballast water after the neutralization is adjusted to be, for example, 0.5 to 3 [mg/L] as Cl₂.

The control part 130 determines whether the discharge of the ballast water comes to an end (step S225). When the control part 130 determines that the discharge does not yet come to an end (NO of step S225), the control part 130 repeats step S223 to step S225. When the control part 130 determines that the discharge comes to an end (YES of step S225), the control part 130 stops the ballast pump 86 and the neutralizer injection pump 124 (step S226) and thereby causes the discharge of the ballast water and the injection of the neutralizer to come to an end. The control part 130 closes the ballast tank exit valve 96 and the ballast water discharge valve 100 (step S227), and thereby causes the water discharge process for the ballast water to come to an end.

In the water discharge process for the ballast water, the control part 130 records the flow amount of the ballast water measured by the ballast flowmeter 90, the flow amount of the neutralizer measured by the neutralizer flowmeter 126, and the concentrations of the TRO measured by the TRO meters 110-1 and 110-2 each with the date and time of the work. The amount of the ballast water in the ballast tank 76 is acquired from the flow amount of the ballast water and the remaining amount of the neutralizer in the neutralizer tank 122 is acquired from the flow amount of the neutralizer. The control part 130 transmits these recorded pieces of equipment data 11 to the control apparatus 14-1.

### <Boiler Equipment 10>

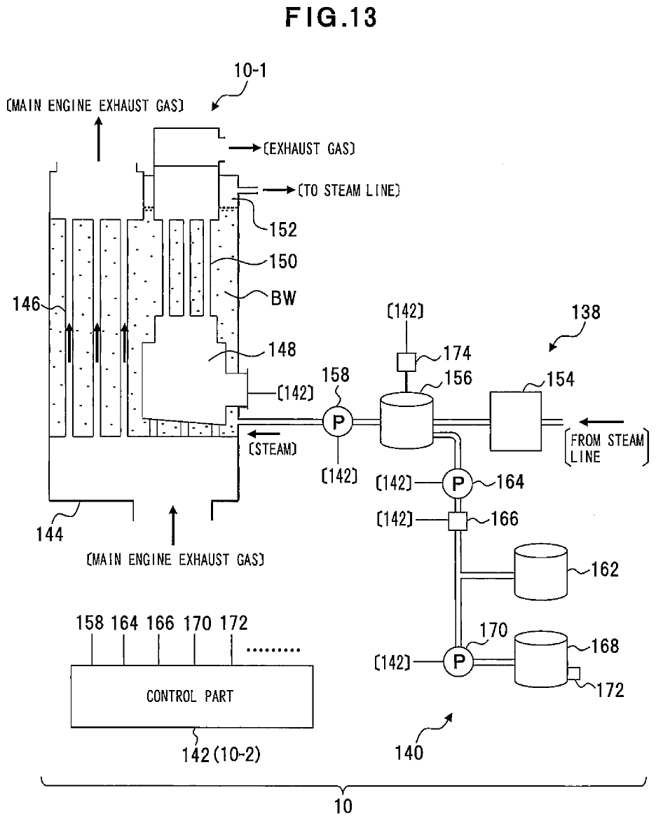

FIG. 13 depicts an example of the boiler equipment. The boiler equipment 10 includes the boiler apparatus 10-1, a circulating apparatus 138, a water supplying apparatus 140, and a control part 142 that is an example of the control part 10-2.

The boiler apparatus 10-1 is, for example, a composite boiler and produces steam from water by heat exchange between the exhaust gas heat discharged from the main engine and the heat generated by oil burning. The boiler apparatus 10-1 includes a housing 144, a main engine smoke pipe 146, an oil burning part 148, and an oil burning smoke pipe 150. The main engine smoke pipe 146, the oil burning part 148, and the oil burning smoke pipe 150 are arranged in the housing 144. The process water BW in the housing 144 is heated by the heat radiation by the main engine smoke pipe 146, the oil burning part 148, and the oil burning smoke pipe 150, and the steam is thereby produced in a steam chamber 152. The produced steam is sent to a steam line of the ship 4.

The circulating apparatus 138 is an example of a means that returns the steam collected from the steam line, to the process water BW and that returns this process water BW to the boiler apparatus 10-1, and includes a water condensing apparatus 154, a water supply tank 156, and a water supply pump 158. The water condensing apparatus 154 cools down the steam collected from the steam line and returns this steam to the process water BW. The process water BW in the water condensing apparatus 154 is sent to the water supply tank 156 and is accumulated in the water supply tank 156. The water supply pump 158 supplies the process water BW in the water supply tank 156 to the boiler apparatus 10-1. In the boiler equipment 10, a circulation path is formed by the boiler apparatus 10-1 and the circulating apparatus 138.

The water supplying apparatus 140 is an example of a process water replenishing means that replenishes the process water BW. The process water BW evaporates into the exterior of the circulation path and reduces over time. The water supplying apparatus 140 replenishes the process water BW and maintains the amount of the process water BW. The water supplying apparatus 140 includes a replenishing water tank 162, a replenishing water pump 164, a flowmeter 166, a drug tank 168, a drug injection pump 170, and a pressure gauge 172. The replenishing water tank 162 accumulates therein, for example, the water produced by the water desalination processing apparatus 12-1. The replenishing water pump 164 is connected to the replenishing water tank 162 and the water supply tank 156, operates in response to, for example, drawdown of the water supply tank 156, and transfers the water in the replenishing water tank 162 to the water supply tank 156. In this case, the drug injection pump 170 is controlled such that the amount of the moved drugs in the drug tank 168 is smaller than the amount of the moved replenishing water, and the drug is thereby prevented from flowing into the replenishing water tank 162. Otherwise, a non-return valve is disposed in the water exit of the replenishing water tank 162 to thereby prevent the drug from flowing into the replenishing water tank 162. The flowmeter 166 is installed between the replenishing water tank 162 and the replenishing water pump 164, and measures the water amount of the water replenished into the circulation path of the boiler equipment 10.

The drug tank 168 accumulates therein the drug that processes the process water BW. This drug suppresses any concentration of the ion component due to reduction of the process water BW, any production of scales, and the like, and is, for example, the scale preventive, the anticorrosive, the sludge dispersant, the disinfectant, or the slime inhibitor. The drug injection pump 170 is connected to the drug tank 168, and a pipe between the replenishing water tank 162 and the flowmeter 166, and transfers the drug in the drug tank 168 to the side of the water supply tank 156. The injection amount of the drug is proportionally controlled by the control part 142 based on, for example, the flow amount of the water measured by the flowmeter 166, and water including the proper amount of drug is replenished into the circulation path of the boiler equipment 10. The pressure gauge 172 is an example of the remaining amount meter that measures the remaining amount of the drug in the drug tank 168 and measures the weights of the drug tank 168 and the drug. The remaining amount of the drug is acquired by subtracting the weight of the drug tank 168 from the measurement result of the pressure gauge 172.

The control part 142 includes, for example, a processor or a programmable logic controller (PLC), operates or stops the oil burning part 148 in accordance with the steam amount, operates or stops the water supply pump 158 in accordance with the water amount of the process water BW in the boiler apparatus 10-1, operate or stops the replenishing water pump 164 in accordance with the water amount in the water supply tank 156, and operates, stops, or adjusts the drug injection pump 170 in accordance with the flow amount of the water measured by the flowmeter 166. The control part 142 records the pieces of equipment data 11 acquired from the measuring devices, the valves, and the pumps each with the date and time of the work, and transmits the pieces of equipment data 11 to the control apparatus 14-1.

A water quality analyzer 174 is an example of an analyzing apparatus that analyzes the water quality of each of the circulation water such as the process water BW of the boiler equipment 10 and other types of water to be measured. The water quality analyzer 174 includes, for example, an absorption photometry part, an electrode measuring part, and an electric conductivity measuring part. The absorption photometry part detects the absorbance of an object to be measured using an absorptiometric method and measures the dissolved component concentration of the object to be measured from this absorbance. The electrode measuring part generates an electric potential corresponding to the ion concentration of the object to be measured and measures the ion concentration of the object to be measured from this electric potential. The electric conductivity measuring part measures the electric conductivity of the object to be measured using, for example, an AC two-electrode method, an AC four-electrode method, or an electromagnetic induction method. When the water quality analyzer 174 is used, not only the water quality analysis for one item but also the water quality analysis for plural items can be executed at one time.

Not limiting to the case for the installation in the boiler equipment 10, the water quality analyzer 174 may, for example, be installed in the ship 4 and measure offline the regularly or irregularly collected process water BW of the boiler equipment 10. In this case, for example, the water quality analyzer 174 is connected to the control apparatus 14-1 by a wire or radio, records the analysis result in the water quality analyzer 174 as the equipment data 11 with the date and time of the work, and transmits the analysis result to the control apparatus 14-1. When the water quality analyzer 174 is used offline, the water quality analyzer 174 only has to transmit the equipment and the water type name information 44 relating to the equipment data 11 together with the equipment data 11, to the control apparatus 14-1. The equipment and the water type name information 44 only has to be input through an input apparatus included in the water quality analyzer 174 or an input apparatus connected to the water quality analyzer 174.

When, for example, a portable small analyzer is used as the water quality analyzer 174, the water quality can be analyzed in the vicinity of the water process equipment. Plural water quality analysis sessions are enabled by one water quality analyzer 174.

### <Process Procedure for Boiler Equipment 10>

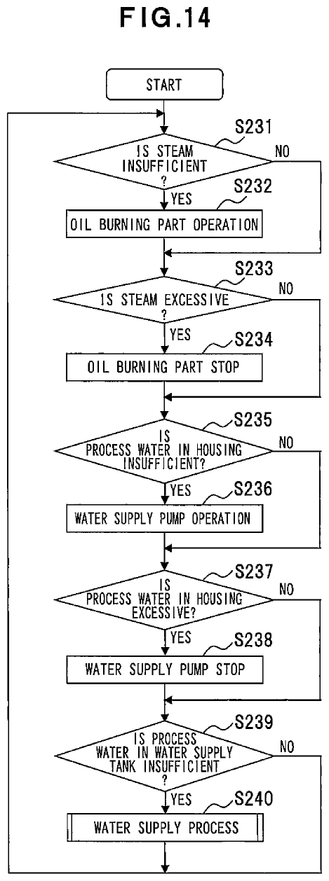

FIG. 14 depicts an example of a process procedure for the boiler equipment 10. This process procedure is executed by the control part 142 of the boiler equipment 10. The processes are an example of the functions executed with the program or the management method. In FIG. 14, “step S” represents a processing stage.

The control part 142 determines whether steam is insufficient from the steam pressure in the circulation path of the boiler equipment 10 (step S231). When the control part 142 determines that the steam is insufficient (YES of step S231), the control part 142 operates the oil burning part 148 (step S232) and produces steam and, when the control part 142 determines that the steam is not insufficient (NO of step S231), the control part 142 does not execute step S232. The insufficiency of the steam only has to be determined based on, for example, determination as to whether the pressure measured by the steam pressure gauge installed on the circulation path of the boiler equipment 10 is lower than the lower limit value of the proper range of the steam pressure.

The control part 142 determines whether steam is excessive from the steam pressure in the circulation path of the boiler equipment 10 (step S233). When the control part 142 determines that the steam is excessive (YES of step S233), the control part 142 stops the oil burning part 148 (step S234) and, when the control part 142 determines that the steam is not excessive (NO of step S233), the control part 142 does not execute step S234. The excessiveness of the steam only has to be determined based on, for example, determination as to whether the pressure measured by the steam pressure gauge exceeds the upper limit value of the proper range of the steam pressure.

The control part 142 determines whether the process water BW in the housing 144 is insufficient (step S235). When the control part 142 determines that the process water BW is insufficient (YES of step S235), the control part 142 operates the water supply pump 158 (step S236) and increases the process water BW in the housing 144 and, when the control part 142 determines that the process water BW is not insufficient (NO of step S235), the control part 142 does not execute step S236. The insufficiency of the process water BW in the housing 144 only has to be determined based on, for example, determination as to whether the water level measured by a water level gauge installed in the housing 144 is lower than the lower limit value of the proper range of the water level of the process water.

The control part 142 determines whether the process water BW in the housing 144 is excessive (step S237). When the control part 142 determines that the process water BW is excessive (YES of step S237), the control part 142 stops the water supply pump 158 (step S238) and, when the control part 142 determines that the process water BW is not excessive (NO of step S237), the control part 142 does not execute step S238. The excessiveness of the process water BW in the housing 144 only has to be determined based on, for example, determination as to whether the water level measured by the water level gauge exceeds the upper limit value of the proper range of the water level of the process water.

The control part 142 determines whether the process water BW in the water supply tank 156 is insufficient (step S239). When the control part 142 determines that the process water BW is insufficient (YES of step S239), the control part 142 executes the water supply process (step S240) and returns to step S231. When the control part 142 determines that the process water BW in the water supply tank 156 is not insufficient (NO of step S239), the control part 142 does not execute step S240 and returns to step S231. The insufficiency of the process water BW in the water supply tank 156 only has to be determined based on, for example, determination as to whether the water level measured by the water level gauge installed in the water supply tank 156 is lower than the lower limit value of the proper range of the water level of the process water.

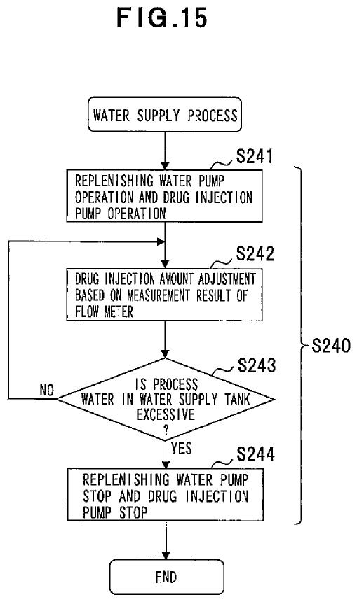

The water supply process (step S240) will be described with reference to FIG. 15. FIG. 15 depicts an example of a process procedure for the water supply process. The processes are an example of the functions executed with the program or the management method. In FIG. 15, “step S” represents a processing stage.

The control part 142 operates the replenishing water pump 164 and the drug injection pump 170 (step S241) and thereby starts injecting the replenishing water and the drug. The control part 142 adjusts the injection amount of the drug based on the measurement result of the flowmeter 166 (step S242). The control part 142 determines whether the process water BW in the water supply tank 156 is excessive (step S243). When the control part 142 determines that the process water BW is excessive (YES of step S243), the control part 142 stops the replenishing water pump 164 and the drug injection pump 170 (step S244), thereby causes the injection of the replenishing water and the drug to come to an end, and thereby causes the water supply process to come to an end. When the control part 142 determines that the process water BW is not excessive (NO of step S243), the control part 142 returns to step S242 and repeats step S242 and step S243. The excessiveness of the process water BW in the water supply tank 156 only has to be determined based on, for example, determination as to whether the water level measured by the water level gauge installed on the water supply tank 156 exceeds the upper limit value of the proper range of the water level of the process water.

In the processes for the boiler equipment 10, the control part 142 records the flow amount measured by the flowmeter 166, the pressure measured by the pressure gauge 172, the remaining amount of the drug acquired from this pressure, the measurement result of the water quality analyzer 174 provided for the water supply tank 156, and the like as pieces of equipment data 11 each with the date and time of the work, and transmits the pieces of equipment data 11 to the control apparatus 14-1.

### <Process Procedure for Water Desalination Processing Apparatus 12-1>

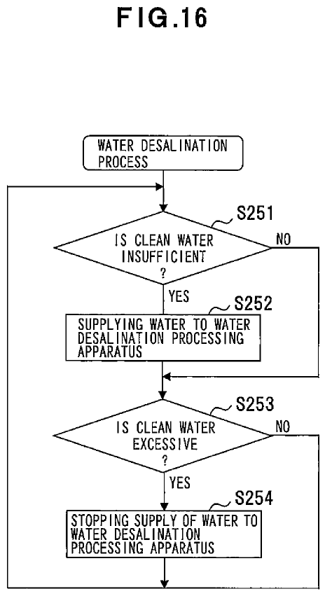

FIG. 16 depicts an example of a process procedure for the water desalination processing apparatus 12-1. This process procedure is executed by the control part 12-2 of the water desalination processing apparatus 12-1. The processes are an example of functions executed with the program or the management method. In FIG. 16, “step S” represents a processing stage.

The control part 12-2 determines whether the clean water is insufficient (step S251). When the control part 12-2 determines that the clean water is insufficient (YES of step S251), the control part 12-2 supplies the used water generated in the ship 4, fresh water such as rain water, or seawater to the water desalination processing apparatus 12-1 (step S252). When the control part 12-2 determines that the clean water is not insufficient (NO of step S251), the control part 12-2 does not execute step S252. The control part 12-2 determines whether the clean water is excessive (step S253). When the control part 12-2 determines that the clean water is excessive (YES of step S253), the control part 12-2 stops the supply of the water to the water desalination processing apparatus 12-1 (step S254) and returns to step S251. When the control part 12-2 determines that the clean water is not excessive (NO of step S253), the control part 12-2 does not execute step S254 and returns to step S251. The insufficiency of the clean water only has to be determined based on, for example, determination as to whether the water level measured by the water level gauge installed on the clean water tank such as each of a storage tank of the domestic-use water, the replenishing water tank 162 of the boiler equipment 10 is lower than the lower limit value of the proper range of the water level of the clean water. The excessiveness of the clean water only has to be determined based on, for example, determination as to whether the water level measured by the water level gauge installed in the clean water tank exceeds the upper limit value of the proper range of the water level of the clean water. The supply of the water to the water desalination processing apparatus 12-1 only has to be executed by, for example, a pump connected to the water desalination processing apparatus 12-1.

In addition to the process procedure, the control part 12-2 analyzes the water quality of the produced clean water regularly or irregularly using the water quality analyzer included in the water desalination processing apparatus 12-1. The water quality analyzer only has to be installed in a place for the water quality analyzer to be able to measure the clean water produced by the water desalination processing apparatus 12-1. The control part 12-2 records the pieces of equipment data 11 such as the operation information on the water desalination processing apparatus 12-1 such as, for example, the operation information on the pump, the analysis result for the produced clean water, and the use amount and the remaining amount of the drug used in the water desalination processing apparatus 12-1 each with the date and time of the work, and transmits the recorded pieces of equipment data 11 to the control apparatus 14-1.

The water quality of the clean water may be measured offline. The water quality analyzer is similar to the water quality analyzer 174 installed in the boiler equipment 10 or the water quality analyzer 174 that measures offline the process water BW of the boiler equipment 10 and will therefore not again be described.

### <Process Procedure for Control Apparatus 14-1>

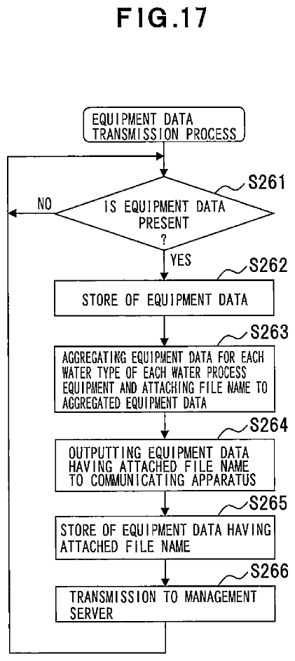

FIG. 17 depicts an example of a process procedure for an equipment data transmission process. This process procedure is an example of an equipment management method of the present invention. This process procedure is executed by execution of the equipment management program readably stored in the memory part 60, by the processor 58 of the control apparatus 14-1 and execution of the communication by the processor of the communicating apparatus 16-1 based on the equipment management program. The processes are an example of the functions executed with the program and the management method. In FIG. 17, “step S” represents a processing stage.

The processor 58 determines whether any reception of the pieces of equipment data 11 is present (step S261). When the processor 58 determines that the reception is present (YES of step S261), the processor 58 stores in the memory part 60 the received pieces of equipment data 11 (step S262). Processor 58 combines to aggregate the pieces of equipment data 11 for each of the water types of each of the pieces of water process equipment, attaches a file name to the aggregated pieces of equipment data 11 (step S263), and thereby produces an equipment data file for transmission to notify the management center 6. The processor 58 refers to, for example, the file name information 46 of the equipment information 34 stored in the memory part 60, and produces the equipment data file for transmission based on the data transmission specification included in the file name information 46. The processor 58 produces the equipment data 11 for transmission so that the data format thereof is recognizable by the management server 23. For example, when the equipment data 11 on the ballast water processing apparatus 8-1 is transmitted, the processor 58 refers to, for example, the ship code “ABCDEFG001” of the ship 4, the equipment name “ballast water process equipment” of the appropriate equipment, and the water type name “ballast water”, and produces the equipment data file for transmission, using the file name “UploadFile001.csv” assigned to these pieces of data. The processor 58 outputs the equipment data 11 for transmission having the file name attached thereto, to the communicating apparatus 16-1 (step S264), and stores the equipment data 11 in the memory part 60 (step S265).

The communicating apparatus 16-1 transmits the equipment data 11 received from the control apparatus 14-1 to the management server 23 (step S266) and returns to step S261.

When the processor 58 determines at step S261 that the reception of the equipment data 11 is not present (NO of step S261), the processor 58 repeats step S261. Due to the execution of the above process procedure, the control apparatus 14-1 and the communicating apparatus 16-1 each transmit plural pieces of equipment data 11 whose measurement times are different from each other and the management server 23 acquires the pieces of equipment data 11.

### <Process Procedure of Management Server 23>

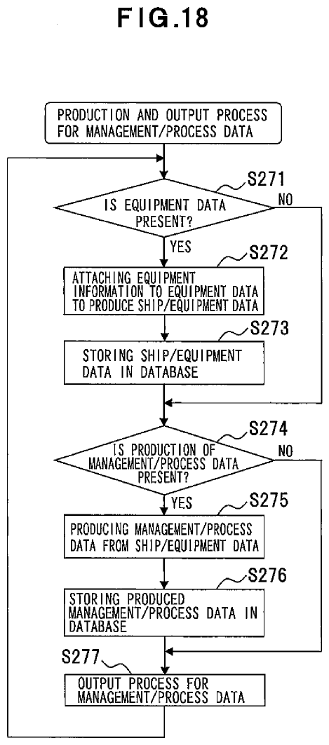

FIG. 18 depicts an example of a process procedures for production and output process for management/process data. The process procedure is an example of the equipment management method of the present invention. This process procedure is executed by execution of the equipment management program readably stored in the memory part 72, by the processor 70 of the management server 23. The processes are an example of the functions executed with the program and the management method. In FIG. 18, “step S” represents a processing stage.

The processor 70 determines whether any reception of the pieces of equipment data 11 is present (step S271). When the processor 70 determines that the reception is present (YES of step S271), the processor 70 attaches the equipment information 34 to the received pieces of equipment data 11, thereby produces the ship/equipment data 28 (step S272), and stores the ship/equipment data 28 in the database 24 (step S273). Processor 70 only has to determine a place for storing the ship/equipment data 28 in the database 24 based on, for example, the file name of the pieces of equipment data 11 received from the communicating apparatus 16-1, or the ship code 36 or the equipment code 40 recorded in the ship/equipment data 28. When the processor 70 determines that the reception of any equipment data 11 is not present (NO of step S271), the processor 70 does not execute step S272 and step S273.

The processor 70 determines whether any production of the management/process data 30 is present (step S274). When the processor 70 determines that the production of the management/process data 30 is present (YES of step S274), the processor 70 produces the management/process data 30 from the ship/equipment data 28 (step S275) and causes the database 24 to store therein the produced management/process data 30 (step S276). The processor 70 only has to produce the management/process data 30 at intervals set in advance each of, for example, six hours, 12 hours, or one day and cause the database 24 to store therein the produced management/process data 30, or only has to produce the management/process data 30 at the time of the reception of the ship/equipment data 28 and cause the database 24 to store therein the produced management/process data 30. When the processor 70 determines that the production of the management/process data 30 is not present (NO of step S274), the processor 70 does not execute step S275 and step S276.

The processor 70 executes an output process for the management/process data 30 (S277) and returns to step S271. The output process for the management/process data 30 only has to be executed at a timing set in advance or in responds to a request by a customer engaged in the ship. The plural pieces of equipment data 11 whose measurement times are different from each other can be stored by repeating step S271 to step S277. The precision of each of the analysis of the pieces of equipment data 11 and the diagnosis of the pieces of water process equipment can be improved or the tendency of the pieces of water process equipment can be presented using a trend graph, by using the plural pieces of equipment data 11 whose measurement times are different from each other.

### Effect of Example 1

(1) The information can be delivered in a short time period because the pieces of equipment data 11 on the pieces of process equipment such as the ballast water processing apparatus 8-1, the boiler apparatus 10-1, and the water desalination processing apparatus 12-1 of the ship 4 on a sea are transmitted to the land through, for example, the satellite communication. The pieces of equipment data 11 transmitted to the management server 23 using the radio communication can immediately be registered in the database 24 and can timely be monitored by the management center 6. The ship can receive in a short time period the management/process data 30 analyzed by the management center 6, and the ship can immediately take a measure based on the management/process data 30.

(2) The management load and the reporting load of the pieces of equipment data 11 on the pieces of water process equipment, and the like on the ship 4 can be reduced and the data provision of the pieces of water process equipment to the customers can be enhanced because the database 24 installed on the land stores therein the ship/equipment data 28 and the management/process data 30, and the management/process data 30 is provided to the customers. When the customers own plural ships 4, the customers can collectively receive the pieces of management/process data 30 on the ships 4 and the data management load on the customers can be reduced.

(3) Because the management server 23 provides the management/process data 30 through the communication network 27, the customers engaged in the ship 4 can utilize this management/process data 30 for various uses. For example, the ship 4 can display the trend graph and the like received from the management server 23 on the displaying part 62 of the control apparatus 14-1 connected to the communicating apparatus 16-1, and can print the trend graph and the like received from the management server 23 using a printing apparatus connected to the control apparatus 14-1. With the displaying or the printing of the management/process data 30, the person in charge of the pieces of water process equipment in the ship 4 refers to the display on the displaying apparatus or printed matters by the printing apparatus, and thereby adjusts the injection amounts of the drugs into the pieces of water process equipment, and equipment adjustment for the pieces of water process equipment is requested to the person in charge of the pieces of water process equipment. With a request for adjustment of the injection amounts of the drugs or the equipment adjustment, the pieces of water process equipment can be maintained in their stable and optimal operation states. The ship owner owning the ship 4 and the ship manager managing the ship 4 can each collectively acquire pieces of information on the plural types of water process equipment of plural ships owned or managed by the ship owners or the ship managers, from the management server 23. Because the management/process data 30 is stored in the database 24, the customers engaged in the ship do not need to individually retain the management/process data 30 and can draw the information from the database 24 when necessary. The customers engaged in the ship can be relieved from the management duties of the management/process data 30 itself.

(4) Because the water quality analysis for each of the pieces of water process equipment is executed on the ship 4 and the data is transmitted to the land using the radio communication, no water sampled from the pieces of water process equipment needs to be sent to the analysis center at any anchorage site of the ship 4 and the analysis results can immediately be acquired.

### Modification Examples

(1) The communication equipment 26 provided by a communication company or the like is used in the above Example while the management center 6 may individually include the communication equipment 26 and the management server 23 may be connected to the communication equipment 26 through a dedicated line. When the communication equipment 26 dedicated to the management center and the dedicated line are used, the risk of information leakage can be reduced between the management server 23 and the communication equipment 26.

(2) The database 24 stores therein the pieces of equipment information 34 of the ship 4 in the above Example while the pieces of equipment information 34 may be stored in the memory part 72 of the management server 23. The storage load on the management server 23 can be reduced when the pieces of equipment information 34 are stored in the database 24, and the risk of communication defect and the like can be reduced when the pieces of equipment information 34 are stored in the management server 23.

(3) The control apparatus 14-1 includes the displaying part 62 and the input part 64 in the above Example while the control apparatus 14-1 may be connected to a displaying apparatus and an input apparatus through the I/O 66. The displaying apparatus only has to have, for example, the function of the displaying part 62 and the input apparatus only has to have, for example, the function of the input part 64.

(4) The management server 23 correlates the equipment data 11 and the equipment information 34 with each other in the above Example. In this case, the data amount transmitted by the ship 4 can be suppressed. The handling of the information is however not limited to that of Example. The ship may correlate in advance the equipment data 11 and the equipment information 34 with each other and may transmit the ship/equipment data 28.

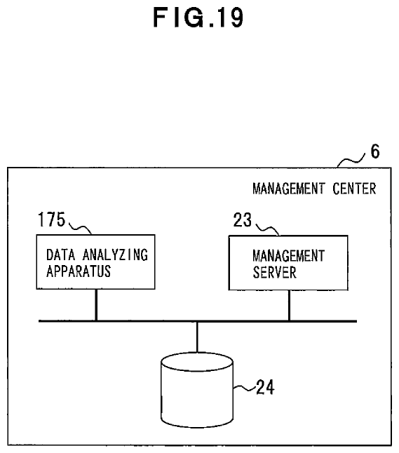

(5) The management server 23 analyzes and diagnoses the data registered in the operation database of the database 24, and processes the data for displaying or printing in the above Example while, as depicted in FIG. 19, a data analyzing apparatus 175 may be included separately from the management server 23 and the function of the management server 23 may be distributed. For example, the data analyzing apparatus 175 may have the data analyzing function, the data diagnosing function, and the data processing function, and may execute these functions instead of the management server 23.

(6) The dissolved component concentration, the ion concentration, and the electric conductivity of each of the process water BW of the boiler equipment 10 and the clean water of the water desalination processing apparatus 12-1 are analyzed in the above Example while a portion of these analysis results may be analyzed, and other water quality items such as, for example, pH, the water temperature, or the drug concentration may be measured.

(7) the control apparatus 14-1 attaches the specific file name to each of the pieces of equipment data 11 on the pieces of water process equipment and the correlating is thereby executed for the equipment data 11 and the water process equipment with each other in the above Example while the correlating is not limited to this correlating using the file name. For example, the pieces of equipment data 11 on the plural pieces of water process equipment may be transmitted with one file name. In this case, the control apparatus 14-1 only has to transmit the equipment data 11 of each of the pieces of water process equipment attaching thereto the ship code 36, the equipment code 40, the water type name information 44, or the like. In this case, the control apparatus 14-1 only has to refer to the equipment information 34 and attach the ship code 36 or the like to the equipment data 11. Otherwise, the ship code 36 or the like may be designated by an operational input on the input part 64 of the control apparatus 14-1. In this case, the equipment data 11 can be correlated with a designated water process equipment or the water type name thereof.

The correlating for the equipment data 11, and the water process equipment or the water type name thereof with each other may be executed using the measuring device such as the water quality analyzer 174. For example, the equipment information 34 may be transmitted in advance from the control apparatus 14-1 to the measuring device and may be stored in the memory of the measuring device. The measuring device may correlate the measured equipment data 11, and the water process equipment or the water type name thereof with each other and may transmit these to the control apparatus 14-1. According to this configuration, the work relating to the correlation of the equipment data 11, and the water process equipment or the water type name thereof is distributed and the load on the control apparatus 14-1 can be reduced.

(8) The management server 23 receives the pieces of equipment data 11 and transmits the management/process data 30 in the above Example while the communication is not limited to the communication through the management server 23. For example, the database 24 may directly be connected to the communication network 27 and the database 24 may directly output the management/process data 30. According to this configuration, the load on the management server 23 is reduced.

(9) For the transmission of the pieces of equipment data 11 by the control apparatus 14-1 and the communicating apparatus 16-1 in the above Example, for example, a transmission interval Tt for the equipment data 11 may be set in the control apparatus 14-1 and the control apparatus 14-1 may collectively transmit the pieces of equipment data 11 acquired during the transmission interval Tt when the transmission interval Tt elapses. The collective transmission of the pieces of equipment data 11 can be facilitated, the number of use sessions of the communication line can be reduced, and improvement of the efficiency of the communication can be facilitated, by the aggregation information process for the pieces of equipment data 11. When the pieces of equipment data 11 are collectively transmitted, pieces of specific equipment data 11 on one piece of equipment may be accumulated for a specific time period and the plural pieces of equipment data 11 acquired during this specific time period may collectively be transmitted, or plural types of equipment data 11 may collectively be transmitted.

(10) The database 24 may further include a user database that defines the correspondence relation among the user name of each of the users using the pieces of water process equipment and the water quality analyzer 174, the user IDs thereof, and the water process equipment for which each of the users analyzes its water. The information in this user database is transmitted to, for example, the control apparatus 14-1 and is stored in the storing part 14-2. When the control apparatus 14-1 accepts the ID of the user using the pieces of water process equipment and the water quality analyzer 174, the control apparatus 14-1 displays the water process equipment or the water quality analyzer 174 correlated with the accepted user ID on the displaying apparatus base on the information in the user database. According to the above configuration, the user can easily learn the water process equipment or the water quality analyzer 174 used by the user by inputting the user ID of the user into the control apparatus 14-1.

(11) The control apparatus 14-1 may display on the displaying part 62 the equipment information 34, the equipment data 11, or the management/process data 30 stored in the storing part 14-2. In this case, the user ID and a password may be set to improve the security. For example, when the control apparatus 14-1 accepts an input of the user ID from the input part 64, the control apparatus 14-1 may prohibit any displaying of the equipment information 34, the equipment data 11, or the management/process data 30 on the water process equipment and the water quality analyzer 174 used by any user other than the user corresponding to the accepted user ID, based on the user data stored in the memory part 60. Due to the prohibition of the displaying, any access by the user to the equipment information on the water process equipment and the water quality analyzer 174 used by another user is blocked and the information security can be enhanced.

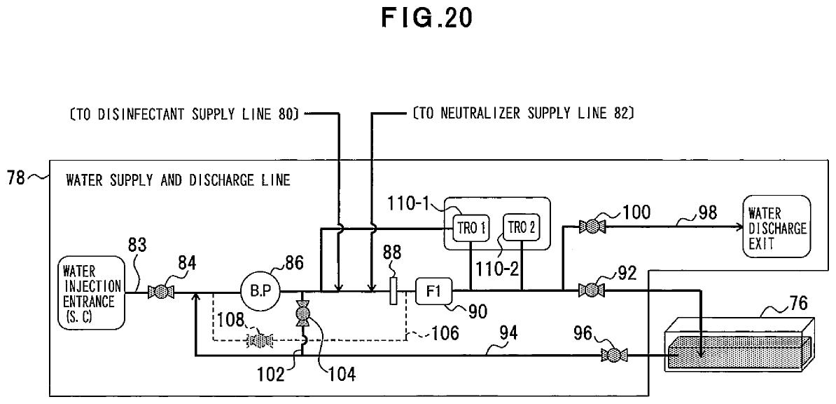

(12) The one end of the branch line 94 is connected to the point between the ballast tank entrance valve 92 and the ballast tank 76 in the above Example while the one end thereof may directly be connected to the ballast tank 76 as depicted in FIG. 20. When the branch line 94 is directly connected to the ballast tank 76, the ballast tank 76, the branch line 94, and the main line 83 form a circulation path that causes the ballast water to circulate therethrough. The ballast water can be circulated when the ballast water injection valve 84 and the ballast water discharge valve 100 are closed, the ballast tank entrance valve 92 and the ballast tank exit valve 96 are opened, and the ballast pump 86 is operated. According to this configuration, in addition to the times when the ballast water is injected and when the ballast water is discharged, when the ballast water is maintained, the ballast water processing apparatus 8-1 can also measure and record the TRO of the ballast water using, for example, the TRO meter 110-1, can inject the disinfectant or the neutralizer based on the measured TRO of the ballast water, and can adjust the TRO of the ballast water.

(13) The management server 23 may have the control authority for the pieces of water process equipment of the ship 4. In this case, the management server 23 only has to output instruction signals to the pieces of water process equipment by radio. The pieces of water process equipment each having the instruction signal received therein through the communicating apparatus 16-1 and the control apparatus 14-1 each only have to adjust the injection of, for example, the disinfectant or the neutralizer in accordance with the instruction signal.

(14) In relation to the water quality management of the ballast water, in the ship 4, the number of viable organisms in the ballast water may be measured and may be transmitted to the management server 23. The number of viable organisms in the ballast water only has to be measured using, for example, a viable organism inspection apparatus. The viable organism inspection apparatus only has to detect the viable organisms defined by the ballast water discharge standards of the above ballast water management standards, that is, the viable organisms that is greater than or equal to 50 micrometers in minimum dimension, and the viable organisms that is less than 50 micrometers in minimum dimension and greater than or equal to 10 micrometers in minimum dimension, distinguishing these from each other and, for example, a ballast water viable organism inspection apparatus approved by IMO only has to be used. For example, a viable organism analyzer in ballast water (model: VOA1000) available from Satake Corporation is usable. The number of the detected viable organisms is used as data indicating the disinfection effect on the viable organisms by the disinfectant, and is stored in the database 24 as disinfection effect check data.

(15) The equipment management program in the above Example may be provided through a communication line such as the Internet or a radio communication line. The equipment management program may be provided through the Internet or a radio communication line or being stored in a recording medium in the state where the equipment management program is encrypted, modulated, or compressed.

(16) The pieces of water process equipment are not limited to the ballast water processing apparatus 8-1, the boiler apparatus 10-1, and the water desalination processing apparatus 12-1, and may be another piece of equipment such as, for example, cooling water equipment or general service water process equipment. For the cooling water equipment, for example, cooling water discharged from a cooling tower, circulation water circulating in the cooling tower, and supply water supplied to the cooling tower are measured and pieces of equipment data 11 thereon are managed. The injection flow amounts and the retention amounts of the drugs such as the slime inhibitor and the scale preventive are managed. These drugs are injected into water passing through the cooling tower. For the general service water process equipment, for example, the water quality of the general service water after the process is measured and the equipment data 11 thereon is managed.

(17) The pieces of measurement information such as the water quality in the ship 4 are used in Example 1 while the seawater may be collected in a port at which the ship 4 anchors or is scheduled to anchor, the environment information such as the measurement information on the seawater may be acquired, this information may be added to the notification information, and these pieces of information may be caused to reflect on the management information.

(18) The TRO meters 110-1 and 110-2 are installed in the water supply and discharge line 78 in Example 1 while the TRO meters 110-1 and 110-2 may be installed in places away from the ballast water processing apparatus 8-1 and may each analyze the collected ballast water.

### Example 2

In Example 2, the equipment management system 2 causes the management/process data 30 provided to the customers engaged in the ship 4 to be displayed on the customer side, or to be printed using a printing apparatus thereon. The equipment management system 2 of Example 2 includes the equipment management system 2 of Example 1. The portions common to Example 1 will not again be described.

In Example 2, the management server 23 provides the management/process data 30 stored in the database 24 to the customers engaged in the ship 4. The management server 23 may processes the management/process data 30 stored in the database 24 and may provide the processed management/process data 30 to the customers. The management server 23 provides the management/process data 30 in response to a request by the customer or automatically to the customers. When the management server 23 provides the management/process data 30 in response to a request by the customer, whether a requesting person is the customer only has to be determined by an authentication process. For example, the user ID and the password of the customer may be stored in the memory part 72 of the management server 23, and the processor 70 of the management server 23 only has to refer to the user ID and the password stored in the memory part 72 and only has to determine whether a user ID input and a password input of the requesting person requesting the management/process data 30 match with those that are referred to. To operate the equipment management system 2, the user ID and the password are set for each of the persons each having the operation authorization for the management server 23.

Authorization levels are set for the user ID and includes, for example, “level 1” at which accesses are enabled to all the pieces of ship/water process information, “level 2” at which provision is allowed of the management/process data 30 of each of the ships 4 owned or managed by an authenticated user, and “level 3” at which provision is allowed of the management/process data 30 of each of the ships 4 relating to an authenticated user. The management server 23 stores these authorization levels in the memory part 72 correlating these levels with the user IDs. Any management/process data 30 not relating to a user accessing the management server 23 is prevented from being provided to the user accessing the management server 23 and a user accessing the management server 23 does not need to exclude information not relating to this user, by setting these authorization levels. For example, because the ship 4 does not receive any management/process data 30 on a ship 4 different therefrom, any wrong handling of data and the like due to an error is prevented.

### <Displaying of Ship/Water Process Information>

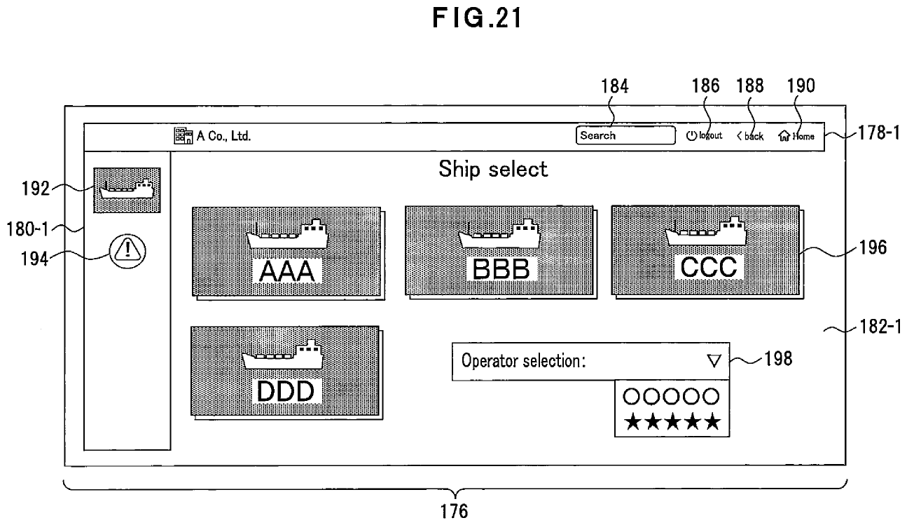

FIG. 21 depicts an example of a ship selection screen. The ship selection screen 176 is caused to be displayed by the management server 23 based on the management/process data 30 or the processed management/process data 30. This screen is displayed after, for example, the authentication process in a log-in screen or a company selection process in a company selection screen. Various types of screen including the ship selection screen 176 are displayed by execution of screen display data stored in the memory part 72 of the management server 23 by the management server 23 or a computer used for the display of this screen. For example, hypertext-type data is usable as the screen display data.

The ship selection screen 176 includes an upper bar 178-1, a side bar 180-1, and a display area 182-1. In the upper bar 178-1, a search button 184, a log-out button 186, a back button 188, and a home button 190 are displayed together with a company name “A Co., Ltd.” selected in the company selection screen. The search button 184 is used for a search in the site. The log-out button 186 is used for logging-out. The back button 188 is used to return to the previous screen. The home button 190 is used to return to the home screen.

In the side bar 180-1, an equipment selection button 192 and an attention button 194 are displayed. The equipment selection button 192 is used to select equipment such as ship equipment, plant equipment, or the like. The attention button 194 represents calling for attention. The ship equipment is selected by the equipment selection button 192 having, for example, a picture of the ship 4 attached thereto. The plant equipment is selected by the equipment selection button 192 having, for example, a picture of a plant attached thereto. In FIG. 21, an example of the equipment selection button 192 having the picture of the ship 4 attached thereto is depicted while the equipment selection button 192 having the picture of a plant attached thereto is not depicted. When no plant equipment managed by the management server 23 is present, no display of the equipment selection button 192 having the picture of a plant attached thereto only has to be displayed. For the selection operation by the various types of selection button, for example, a touch operation onto a touch panel, or a clicking operation by a mouse only has to be used.

In the upper portion of the display area 182-1 of the ship selection screen 176, a title “Ship select” representing the selection of the ship 4 is displayed. On the lower side of this title, ship selection buttons 196 of the ships 4 relating to the company displayed in the upper bar 178-1 are displayed. Each of the ship selection buttons 196 has a figure representing a ship and a ship name “AAA”, “BBB”, “CCC”, or “DDD” displayed therein. In the display area 182-1, an operator selection display 198 is further displayed. Selection of an inverted-triangle mark on the right-side end of the operator selection display 198 causes the management server 23 to display registered operator names on the lower side of the operator selection display 198. Selection of an operator name determines an operator. In this ship selection screen 176, a specific ship selection button 196 is selected. In response to the selection of the ship selection button 196, the management server 23 switches the screen to a selection screen for the water process equipment of the selected ship 4.

In the selection screen for the water process equipment, selection buttons representing, for example, the ballast water processing apparatus 8-1, the boiler apparatus 10-1, and the water desalination processing apparatus 12-1 only have to be displayed. Selection of these selection buttons causes the management server 23 to switch the display to the display of the management/process data 30 of the selected water process equipment.

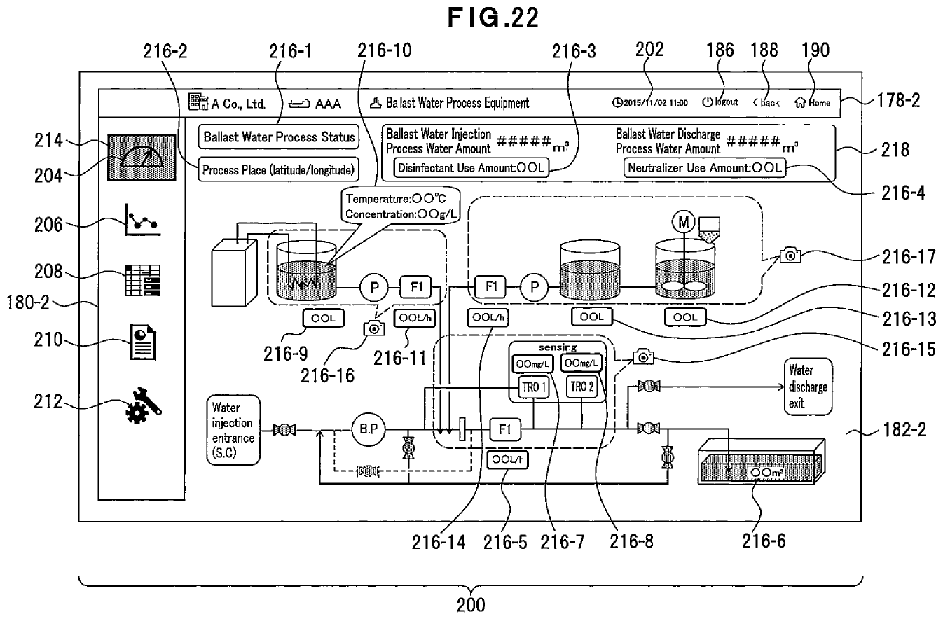

FIG. 22 depicts an example of a water process status screen. This water process status screen 200 is an example of the display of the management/process data 30 and, when the ballast water processing apparatus 8-1 is selected in the selection screen for the water process equipment, the management server 23 displays this screen. The water process status screen 200 depicted in FIG. 22 displays the water process status of the ballast water processing apparatus 8-1.

The water process status screen 200 includes an upper bar 178-2, a side bar 180-2, and a display area 182-2. In the upper bar 178-2, a time of day display 202, the log-out button 186, the back button 188, and the home button 190 are displayed together with the company name “A Co., Ltd.”, the ship name “AAA”, and the water process equipment name “Ballast Water Process Equipment”.

In the side bar 180-2, an equipment status selection button 204, a progress information selection button 206, a drug information selection button 208, a reporting document selection button 210, and a setting change button 212 are displayed. The equipment status selection button 204 is used for the display of the water process status in the water process equipment. The progress information selection button 206 is used for the display of a change associated with the elapse of time of the equipment data 11 relating to the water process equipment. The drug information selection button 208 is used for the display of the information on the drugs used by the water process equipment. The reporting document selection button 210 is used for the display or printing of the reporting document such as a daily report, a weekly report, a monthly report, and the like relating to the sailing of the ship 4. The setting change button 212 is used for a setting change of the water process status screen 200. A button currently selected has a display process 214 attached thereto and the fact that this button is currently selected is thereby represented. The display process 214 is executed by, for example, a coloring process, a hatching process of attaching a pattern such as slashes, halftone screening, or dots, a framing process of attaching an emphasizing frame around the button, or any combination of these.

On the left side in the upper portion of the display area 182-2 of the water process status screen 200, selection buttons 216-1 and 216-2 are displayed. The selection button 216-1 has “Ballast Water Process Status” displayed therein. Selection of this selection button 216-1 causes the process status of the ballast water to be displayed in the display area 182-2. The selection button 216-2 has “Process Place (latitude/longitude)” displayed therein. Selection of this selection button 216-2 causes the information on the latitude and the longitude to be displayed as the information on the place for the ballast water to be processed. The information on the latitude and the longitude can be acquired by referring to the sailing schedule data of each of the ships 4 and the process time of day of the ballast water of the ship 4 managed by the management server 23. This information may also be displayed by acquiring the position information by the global positioning system (GPS) or the like from the ship 4 when the management server 23 receives the pieces of equipment data 11.

On the right side in the upper portion of the display area 182-2, a display frame 218 is set. In this display frame 218, the display “Ballast Water Injection Process Water Amount” is displayed together with the water injection process water amount of the ballast water, and the display “Ballast Water Discharge Process Water Amount” is displayed together with the water discharge process water amount of the ballast water. In the display frame 218, selection buttons 216-3 and 216-4 are displayed. The selection button 216-3 causes the recent equipment data value relating to the ballast water injection process water amount to be displayed. The selection button 216-4 causes the recent equipment data value relating to the ballast water discharge process water amount to be displayed. The selection button 216-3 has the disinfectant use amount displayed thereon together with the display “Disinfectant Use Amount:”. The selection button 216-4 has the neutralizer use amount displayed thereon together with the display “Neutralizer Use Amount:”.

In the central portion and the lower portion of the display area 182-2, selection buttons 216-5 and 216-6 to 216-14 and selection buttons 216-15, 216-16, and 216-17 are displayed together with an equipment configuration diagram of the ballast water processing apparatus 8-1. The selection buttons 216-5 and 216-6 to 216-14 each cause the recent equipment data value of the water process equipment to be displayed. The selection buttons 216-15, 216-16, and 216-17 each cause an image of the water process equipment to be displayed in the screen. The selection button 216-5 is displayed corresponding to the ballast flowmeter display and is displayed in, for example, the lower portion. The selection button 216-5 has the measured flow amount of the ballast flowmeter 90 displayed therein. The selection button 216-6 is displayed corresponding to the ballast tank display and is displayed, for example, being superimposed on the ballast tank display. The selection button 216-6 has the flow amount of the ballast water in the ballast tank 76 displayed therein. The selection button 216-7 is displayed corresponding to the TRO meter display (TRO 1) and is displayed in, for example, the upper portion. The selection button 216-7 has the measured TRO value of the TRO meter 110-1 displayed therein. The selection button 216-8 is displayed corresponding to the TRO meter display (TRO 2) and is displayed in, for example, the upper portion. The selection button 216-8 has the measured TRO value of the TRO meter 110-2 displayed therein. The selection button 216-9 is displayed corresponding to the disinfectant tank display and is displayed in, for example, the lower portion. The selection button 216-9 has the remaining amount of the disinfectant in the disinfectant tank 114 displayed therein. The selection button 216-10 is displayed corresponding to the disinfectant tank display and is displayed in, for example, the upper right portion. The selection button 216-10 has the temperature and the concentration of the disinfectant in the disinfectant tank 114 displayed therein. The selection button 216-11 is displayed corresponding to the disinfectant flowmeter display and is displayed in, for example, the lower portion. The selection button 216-11 has the measured flow amount of the disinfectant flowmeter 118 displayed therein. The selection button 216-12 is displayed corresponding to the neutralizer dissolving tank display and is displayed in, for example, the lower portion. The selection button 216-12 has the remaining amount of the neutralizer in the neutralizer dissolving tank 120 displayed therein. The selection button 216-13 is displayed corresponding to the neutralizer tank display and is displayed in, for example, the lower portion. The selection button 216-13 has the remaining amount of the neutralizer in the neutralizer tank 122 displayed therein. The selection button 216-14 is displayed corresponding to the neutralizer flowmeter display and is displayed in, for example, the lower portion. The selection button 216-14 has the measured flow amount of the neutralizer flowmeter 126 displayed therein. The selection button 216-15 includes a balloon portion surrounding a mixer display, a ballast flowmeter display, and TRO meter displays (TRO 1 and TRO 2), and a camera display. The selection button 216-16 includes a balloon portion surrounding a disinfectant tank display, a disinfectant injection pump display, and a disinfectant flowmeter display, and a camera display. The selection button 216-17 includes a balloon portion surrounding a neutralizer dissolving tank display, a neutralizer tank display, a neutralizer injection pump display, and a neutralizer flowmeter display, and a camera display.

In the equipment configuration diagram of the ballast water processing apparatus 8-1 displayed in the display area 182-2, the management server 23 distinguishably displays, for example, the opening or closing state of each of the valves, water passing state of the ballast water, and the operation state of each of the devices using colors and variations of display, and displays the flows of the ballast water, the disinfectant, and the neutralizer using animations. Progress of easy understanding of the process state is facilitated by using the colors, and variations of the display.

In accordance with the selection of any of the selection buttons 216-3 to the selection button 216-14, the management server 23 causes the time elapse information relating to the selected equipment data 11 to be displayed as, for example, a graph in the display area 182-2. With the selection buttons 216-3 to 216-14, the information can easily be switched to the information desired to be acquired by a simple operation. In accordance with the selection of the selection button 216-15, 216-16, or 216-17, the management server 23 displays an image of the water processing apparatuses in a selected range in the display area 182-2. For example, in response to the selection of the selection button 216-15, 216-16, or 216-17, the management server 23 causes shot image by the shooting apparatus 132-1, 132-2, or 132-3 to be displayed in the display area 182-2. The image to be displayed only has to be a real-time image, a live image, or a recorded image. With these images, monitoring for, for example, any leakage of the drugs can be executed, and the equipment states can be shared between the ship 4 and the land using the images by installing the shooting apparatuses 132-1, 132-2, and 132-3.

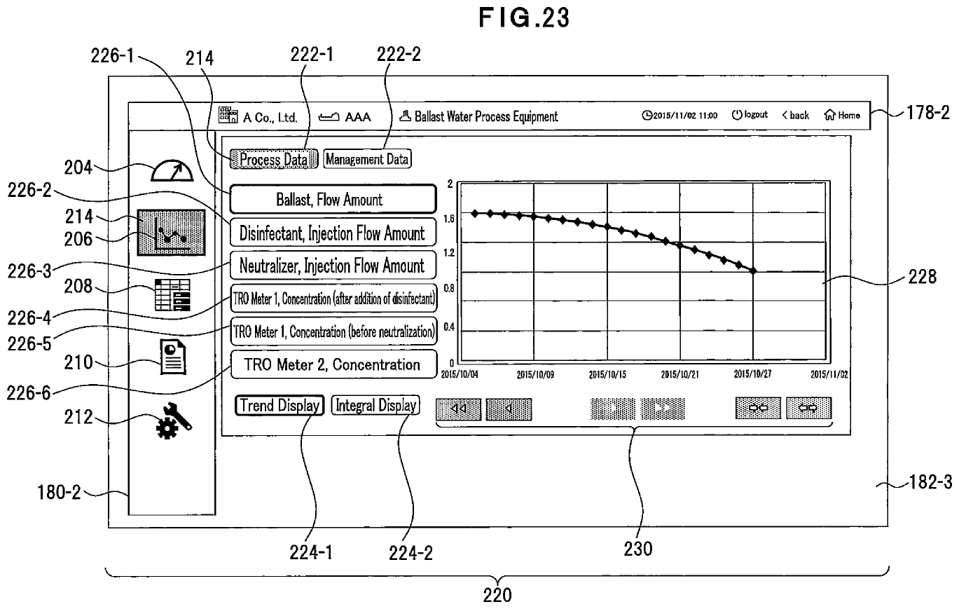

FIG. 23 depicts an example of a trend display screen. The trend display screen 220 is an example of the display of the management/process data 30 and is displayed by the management server 23, for example, when the selection button 216-5 corresponding to the ballast flowmeter display is selected in the water process status screen 200. The trend display screen 220 includes the upper bar 178-2, the side bar 180-2, and a display area 182-3. The upper bar 178-2 and the side bar 180-2 are similar to the upper bar 178-2 and the side bar 180-2 in the water process status screen 200. In the trend display screen 220, the display process 214 is attached to the progress information selection button 206.

In the upper-left portion of the display area 182-3 of the trend display screen 220, data switch buttons 222-1 and 222-2 each used for switching the data type are displayed. The data switch button 222-1 has the display “Process Data” displayed therein and the data switch button 222-2 has the display “Management Data” displayed therein. Selection of the data switch button 222-1 causes the management server 23 to switch the display in the display area 182-3 to the process data, and selection of the data switch button 222-2 causes the management server 23 to switch the display in the display area 182-3 to the management data. In the display area 182-3 depicted in FIG. 23, the display process 214 is attached to the data switch button 222-1 to indicate that the process data is currently selected. For the selection operation for the various types of switch button, for example, a touch operation onto a touch panel, or a clicking operation by a mouse only has to be used.

In the lower-left portion of the display area 182-3, display switch buttons 224-1 and 224-2 each used for switching the display type are displayed. The display switch button 224-1 has the display “Trend Display” displayed therein and the display switch button 224-2 has the display “Integral Display” displayed therein. Selection of the display switch button 224-1 causes the management server 23 to switch the display in the display area 182-3 to the trend display that shows the tendency of the data, and selection of the display switch button 224-2 causes the management server 23 to switch the display in the display area 182-3 to the integral display that shows the integral value of the data. In the display area 182-3 depicted in FIG. 23, the display process 214 is attached to the display switch button 224-1 to indicate that the trend display is currently selected.

In the center-left portion of the display area 182-3 having the selected process data and the selected trend display, selection buttons 226-1 and 226-2 to 226-6 each used for the display of the equipment data 11 including the tendency information are displayed. The selection button 226-1 has the display “Ballast, Flow Amount” displayed therein, the selection button 226-2 has the display “Disinfectant, Injection Flow Amount” displayed therein, the selection button 226-3 has the display “Neutralizer, Injection Flow Amount” displayed therein, the selection button 226-4 has the display “TRO Meter 1, Concentration (after addition of disinfectant)” displayed therein, the selection button 226-5 has the display “TRO Meter 1, Concentration (before neutralization)” displayed therein, and the selection button 226-6 has the display “TRO Meter 2, Concentration” displayed therein. In response to selection of any of these selection buttons 226-1 and 226-2 to 226-6, the management server 23 displays the equipment data 11 that corresponds to the display of the selected one of the selection buttons 226-1 and 226-2 to 226-6, in a data area 228 in the central portion and on the right side of the display area 182-3. In the display area 182-3 depicted in FIG. 23, the display process 214 is attached to the selection button 226-1 and the trend of the equipment data 11 of the ballast flow amount is displayed. The equipment data 11 corresponding to the selected display is displayed in, for example, a graph whose axis of ordinate represents the value of the equipment data 11 and whose axis of abscissa represents the date and the time of day. In the lower portion of the data area 228, an operation part 230 is displayed and the range of each of the date and the time of day of the data to be displayed can be changed by an operation on the operation part 230.

This trend display screen 220 can be displayed by selecting the progress information selection button 206 in the side bar 180-2 and thereafter selecting the data switch button 222-1, the display switch button 224-1, and the selection button 226-1 that are displayed in the display area 182-3.

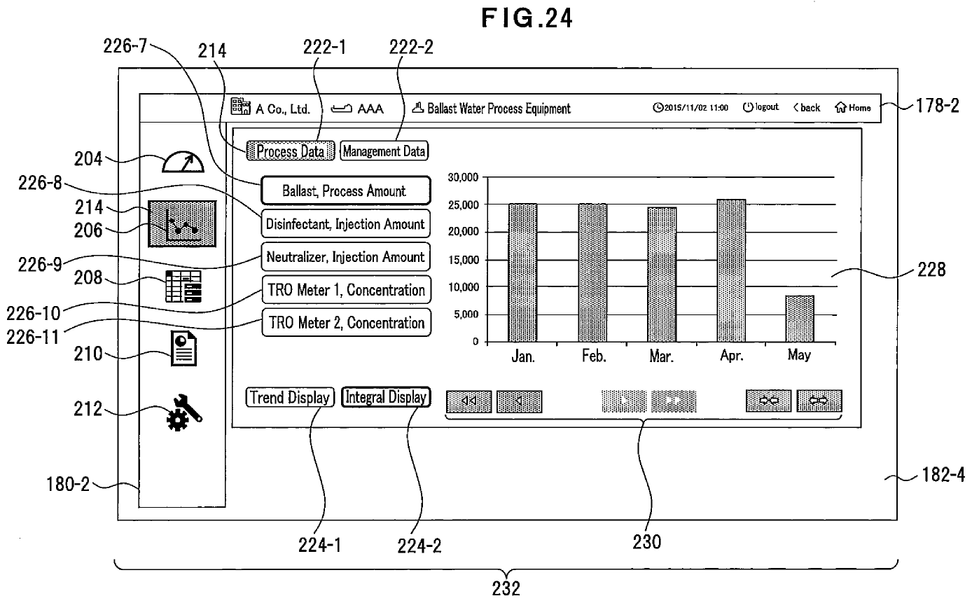

FIG. 24 depicts an example of an integral display screen. The integral display screen 232 is an example of the display of the management/process data 30 and is displayed by the management server 23 when, for example, the selection button 216-5 corresponding to the ballast flowmeter display is selected in the water process status screen 200. When the trend display is currently selected, the screen can be switched to the integral display screen by selecting the display switch button 224-2.

In the integral display screen 232, selection buttons 226-7 and 226-8 to 226-11 each used for the display of the equipment data 11 including the integral information are displayed. The selection button 226-7 has the display “Ballast, Process Amount” displayed therein, the selection button 226-8 has the display “Disinfectant, Injection Amount” displayed therein, the selection button 226-9 has the display “Neutralizer, Injection Amount” displayed therein, the selection button 226-10 has the display “TRO Meter 1, Concentration” displayed therein, and the selection button 226-11 has the display “TRO Meter 2, Concentration” displayed therein. In response to selection of any one of these selection buttons 226-7 and 226-8 to 226-11, the management server 23 displays the equipment data 11 that corresponds to the selected display in the data area 228 in a display area 182-4. In the display area 182-4 depicted in FIG. 24, the display process 214 is attached to the selection button 226-7 and the data of the ballast process amount is displayed as the integral amount. The other displays are similar to those of the trend display screen 220 and will not again be described.

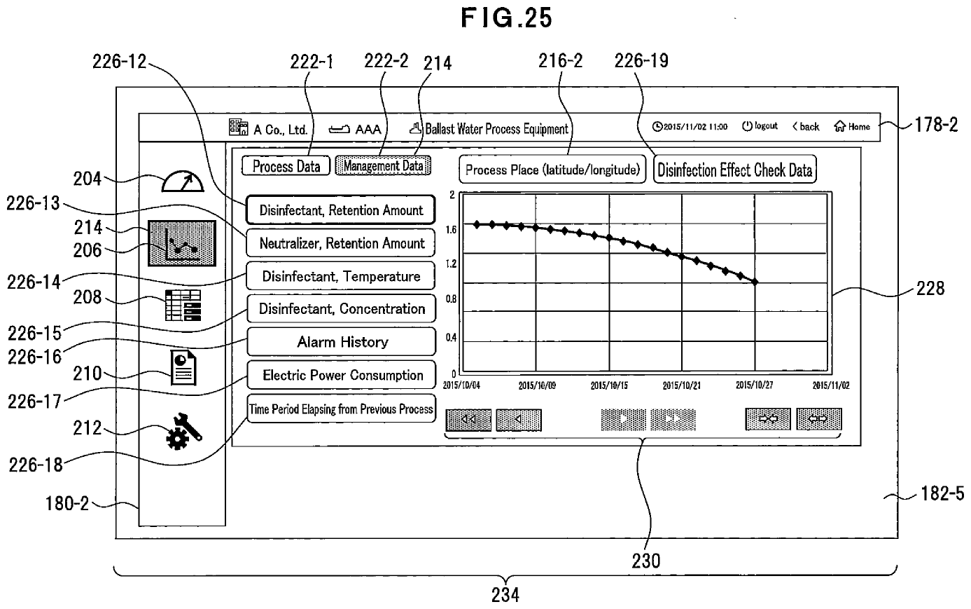

FIG. 25 depicts an example of a management data display screen. The management data display screen 234 is an example of the display of the management/process data 30 and is displayed by the management server 23 in, for example, the water process status screen 200 when the selection button 216-9 corresponding to the disinfectant tank display is selected. When the process data is currently selected, selection of the data switch button 222-2 can cause the screen to be switched to the management data display screen 234.

The management data display screen 234 includes the upper bar 178-2, the side bar 180-2, and the display area 182-5. The upper bar 178-2 and the side bar 180-2 are similar to the upper bar 178-2 and the side bar 180-2 in the trend display screen 220.

In the upper-left portion of the display area 182-5 of the management data display screen 234, the data switch buttons 222-1 and 222-2 are displayed and, in the center-left portion and the lower-left portion of the display area 182-5, selection buttons 226-12 and 226-13 to 226-18 are displayed. The selection button 226-12 has the display “Disinfectant, Retention Amount” displayed therein. The selection button 226-13 has the display “Neutralizer, Retention Amount” displayed therein. The selection button 226-14 has the display “Disinfectant, Temperature” displayed therein. The selection button 226-15 has the display “Disinfectant, Concentration” displayed therein. The selection button 226-16 has the display “Alarm History” displayed therein. The selection button 216-17 has the display “Electric Power Consumption” displayed therein. The selection button 216-18 has the display “Time Period Elapsing from Previous Process” displayed therein. In response to selection of anyone of these selection buttons 226-12 and 226-13 to 226-18, the management server 23 displays the equipment data 11 that corresponds to the display of the selected one of the selection buttons 226-12 and 226-13 to 226-18, in the data area 228 in the central portion and in the right portion of the display area 182-5. In the display area 182-5 depicted in FIG. 25, the display process 214 is attached to the selection button 226-12 and the retention amount of the disinfectant is displayed. The equipment data 11 corresponding to the selected display is displayed in, for example, a graph whose axis of ordinate represents the value of the equipment data 11 and whose axis of abscissa represents the date and the time of day. On the lower side of the data area 228, the operation part 230 is displayed and the range of each of the date and the time of day of the data to be displayed can be changed by an operation on the operation part 230.

In the upper portion of the data area 228, a selection button 216-2 and a selection button 226-19 are displayed. The selection button 216-2 is similar to the selection button 216-2 displayed in the water process status screen 200. The selection button 226-19 has the display “Disinfection Effect Check Data” displayed therein. In response to selection of anyone of these selection buttons 216-2 and 226-19, the management server 23 displays the data of the displayed item of the selected one of the selection buttons 216-2 and 226-19 in the data area 228.

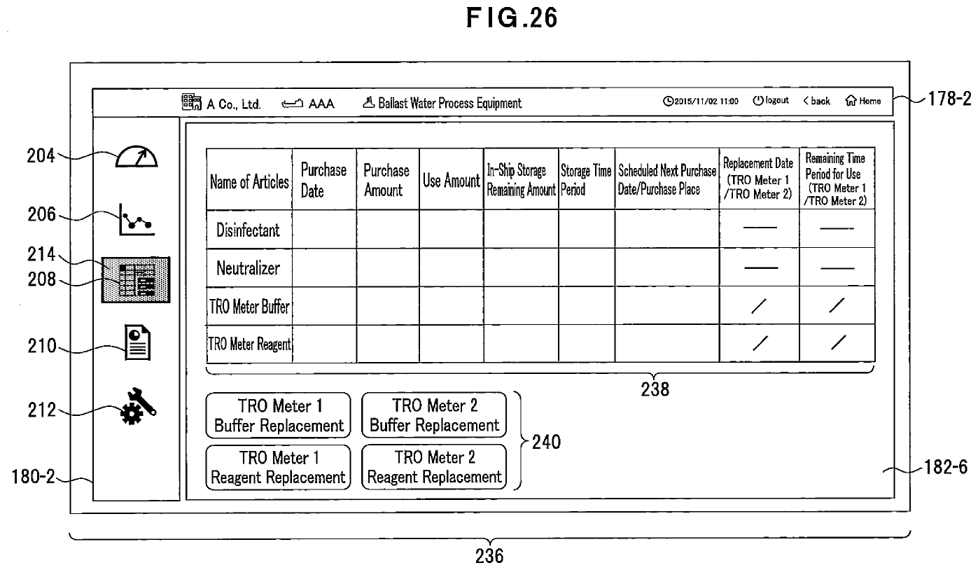

FIG. 26 depicts an example of a drug information display screen. The drug information display screen 236 is an example of the display of the management/process data 30 and can be displayed by, for example, selecting the drug information selection button 208. The drug information display screen 236 includes the upper bar 178-2, the side bar 180-2, and a display area 182-6. The upper bar 178-2 and the side bar 180-2 are similar to the upper bar 178-2 and the side bar 180-2 in the water process status screen 200. In the drug information display screen 236, the display process 214 is attached to the drug information selection button 208.

In the upper portion and the central portion of the display area 182-6, a drug information table 238 is displayed. The drug information table 238 displays therein various types of management item and the data thereon of the drugs used in the pieces of water process equipment. The drug information table 238 displays therein the pieces of information on the management items of “Purchase Date”, “Purchase Amount”, “Use Amount”, “In-Ship Storage Remaining Amount”, “Storage Time Period”, and “Scheduled Next Purchase Date/Purchase Place” for each of the drugs having the names of “Disinfectant”, “Neutralizer”, “TRO Meter Buffer”, and “TRO Meter Reagent”. The management items of “Replacement Date” and “Remaining Time Period for Use” are displayed for each of the drugs having the names of “TRO Meter Buffer” and “TRO Reagent”. In the drug information table 238, the pieces of data of “Replacement Date” and “Remaining Time Period for Use” of the buffer (a buffer solution), and the pieces of data of “Replacement Date” and “Remaining Time Period for Use” of the reagent are displayed for each of the TRO meters 110-1 and 110-2.

In the lower portion of the display area 182-6, a drug replacement button 240 instructing the replacement end of the drug is displayed. This drug replacement button 240 is operated when the drug is replaced and instructs the replacement end to the management server 23 in response to an operation thereof. When the management server 23 receives the replacement end instruction, the management server 23 updates the data in the replacement date column in the drug information table 238 to the date of reception of this instruction. The replacement date in the drug information table 238 is updated in response to the operation of the drug replacement button 240. The remaining time period for use is updated relative to the replacement date. For example, the remaining time period for use of the TRO meter reagent is set to be, for example, three months from the replacement date and, when the replacement date is updated, the remaining time period for use is also updated.

For the operation of the drug replacement button 240, for example, a touch operation onto a touch panel, or a clicking operation by a mouse only has to be used. The various types of screen including the drug information display screen 236 are usable for updating of the equipment data 11 or the management/process data 30 as the replacement end instruction by the drug replacement button 240. For the buffer replacement or the reagent replacement, the data can be updated by clicking once without any inputting of the replacement date, and the work by the shipmen can be reduced.

### <Reporting of Ship/Water Process Information>

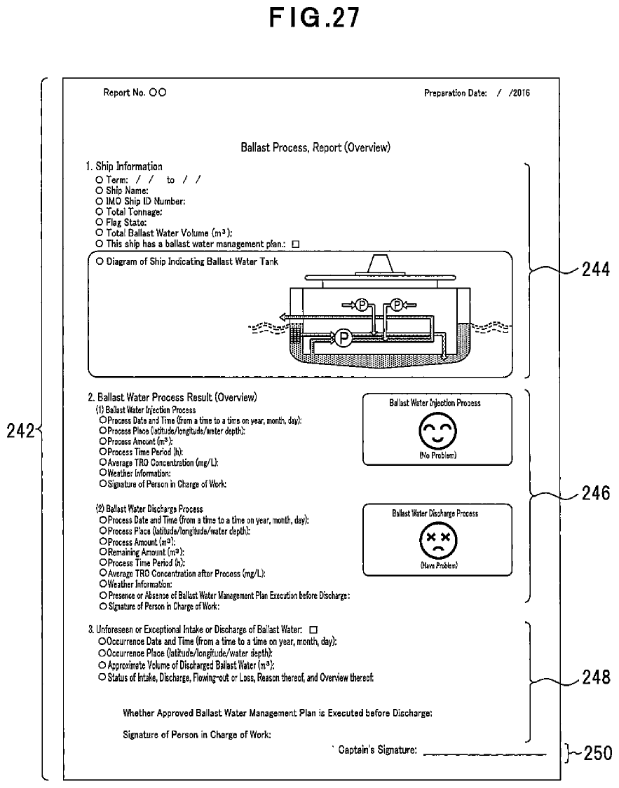

FIG. 27 depicts an example of an overview reporting document. This reporting document is an example of the reporting document that is prepared based on the management/process data 30, and is displayed or printed by, for example, selecting the reporting document selection button 210.

The overview reporting document 242 includes a ship information column 244, a ballast water process column 246, a miscellaneous item column 248, and a captain's signature column 250 together with the report number description, preparation date description, and a title “Ballast Process, Report (Overview)”. The overview reporting document 242 is prepared, for example, every one day regardless of the request for displaying or printing. When a new overview reporting document 242 is prepared, the management server 23 refers to the report number of the last overview reporting document 242 stored in the memory part 72, issues a new report number, displays the new report number in the new overview reporting document 242, and stores in the memory part 72 as the latest report number. Relating to this report number, the management server 23 may refer to the report number of the overview reporting document 242 stored in the database 24 as the management/process data 30 and may issue the new report number. When the management server 23 prepares the new reporting document, the management server 23 refers to the date information of the management server 23 and displays the date together with the preparation date description.

The ship information column 244 includes description columns “Term”, “Ship Name”, “IMO Ship ID Number”, “Total Tonnage”, “Flag State”, “Total Ballast Water Volume (m³)”, “This ship has a ballast water management plan.”, and “Diagram of Ship Indicating Ballast Water Tank”, together with a title “1. Ship Information”. For example, the management server 23 automatically fills these description columns based on the data in the database 24. These description columns are filled in accordance with, for example, the form of the “ballast water record book” stipulated in the ballast water management convention.

The ballast water process column 246 includes titles “2. Ballast Water Process Result (Overview)”, “(1) Ballast Water Injection Process”, and “(2) Ballast Water Discharge Process”. Relating to the title “(1) Ballast Water Injection Process”, the ballast water process column 246 includes description columns “Process Date and Time (from a time to a time on year, month, day)”, “Process Place (latitude/longitude/water depth)”, “Process Amount (m³)”, “Process Time Period (h)”, “Average TRO Concentration (mg/L)”, “Weather Information”, and “Signature of Person in Charge of Work”. Relating to the title “(2) Ballast Water Discharge Process”, the ballast water process column 246 includes description columns “Process Date and Time (from a time to a time on year, month, day)”, “Process Place (latitude/longitude/water depth)”, “Process Amount (m³)”, “Remaining Amount (m³)”, “Process Time Period (h)”, “Average TRO Concentration after Process (mg/L)”, “Weather Information”, “Presence or Absence of Ballast Water Management Plan Execution before Discharge” and “Signature of Person in Charge of Work”. For example, the management server 23 automatically fills these description columns based on the data in the database 24. These description columns are filled in accordance with, for example, the form of the ballast water record book.

This ballast water process column 246 further includes a result display column showing the result of the ballast water injection process and a result display column showing the result of the ballast water discharge process. These result display columns each include, for example, a face mark, and each show presence or absence of any problem of the ballast water injection process or the ballast water discharge process using the expression on the face. These result display columns are displayed by, for example, the management server 23. The management server 23 determines whether any abnormality is present in the item described in each of the description columns. The management server 23 displays the display representing absence of any problem when the management server 23 determines that no abnormality is present, and displays the display representing presence of a problem when the management server 23 determines that abnormality is present.

The miscellaneous item column 248 is a description column relating to an unforeseen or exceptional intake or discharge of the ballast water, and includes description columns “Occurrence Date and Time (from a time to a time on year, month, day)”, “Occurrence Place (latitude/longitude/water depth)”, “Approximate Volume of Discharged Ballast Water (m³)”, “Status of Intake, Discharge, Flowing-out or Loss, Reason thereof, and Overview thereof”, “Whether Approved Ballast Water Management Plan is Executed before Discharge”, and “Signature of Person in Charge of Work”, together with a square-shaped description column with which occurrence of such intake or discharge of the ballast water is indicated by putting a check therein. These items are described by, for example, the person in charge of the work of the ship 4. These description columns are filled in accordance with, for example, the form of the ballast water record book. The management server 23 may automatically fill these description columns based on the data in the database 24.

The captain's signature column 250 includes a description column “Captain's Signature”. The captain reviews this “Ballast Process, Report (Overview)” and signs in the description column “Captain's Signature”. This overview reporting document 242 is usable as the ballast water record book stipulated in the ballast water management convention and as the journal of the ship 4.

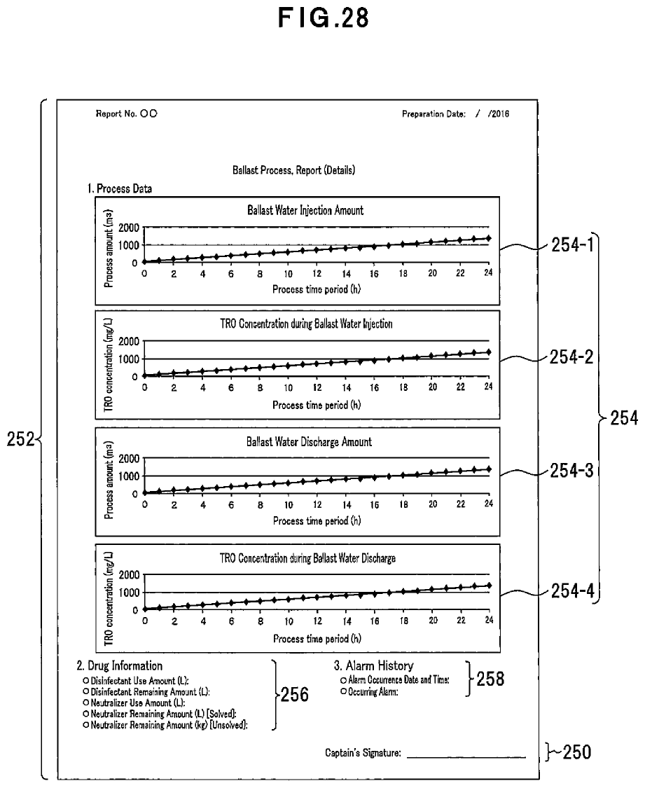

FIG. 28 depicts an example of a detailed reporting document. This reporting document is an example of the reporting document prepared based on the management/process data 30 and is, for example, displayed following the overview reporting document 242 or printed following the overview reporting document 242.

The detailed reporting document 252 includes a process data column 254, a drug information column 256, an alarm history column 258, and the captain's signature column 250, together with the report number description, the preparation date description, and the title “Ballast Process, Report (Details)”.

The process data column 254 includes display columns 254-1, 254-2, 254-3, and 254-4, together with a title “1. Process Data”. The display column 254-1 is, for example, a display column “Ballast Water Injection Amount” and is a graph whose axis of ordinate represents the process amount (m³), whose axis abscissa represents the process time period (h), and that displays, for example, the change of the ballast water injection amount. The display column 254-2 is, for example, a display column “TRO Concentration during Ballast Water Injection” and is a graph whose axis of ordinate represents the TRO concentration (mg/L), whose axis abscissa represents the process time period (h), and that displays, for example, the change of the TRO concentration during the ballast water injection. The display column 254-3 is, for example, a display column “Ballast Water Discharge Amount” and is a graph whose axis of ordinate represents the process amount (m³), whose axis abscissa represents the process time period (h), and that displays, for example, the change of the ballast water discharge amount. The display column 254-4 is, for example, a display column “TRO Concentration during Ballast Water Discharge” and is a graph whose axis of ordinate represents the TRO concentration (mg/L), whose axis abscissa represents the process time period (h), and that displays, for example, the change of the TRO concentration during the ballast water discharge. The process time period display of each of these display columns 254-1, 254-2, 254-3, and 254-4 is displayed for, for example, 24 hours from 0:00 to 24:00 on the day of the preparation of this detailed reporting document 252. The management server 23 automatically displays these display columns 254-1, 254-2, 254-3, and 254-4 based on the management/process data 30 of the database 24.

The drug information column 256 includes description columns “Disinfectant Use Amount (L)”, “Disinfectant Remaining Amount (L)”, “Neutralizer Use Amount (L)”, “Neutralizer Remaining Amount (L) [Solved]”, and “Neutralizer Remaining Amount (kg) [Unsolved]”, together with a title “2. Drug Information”. The management server 23 automatically describes in the description columns “Disinfectant Use Amount (L)”, “Disinfectant Remaining Amount (L)”, “Neutralizer Use Amount (L)”, and “Neutralizer Remaining Amount (L) [Solved]” based on the data in the database 24. The description column “Neutralizer Remaining Amount (kg) [Unsolved]” is described by, for example, the person in charge of the work of the ship 4. The description column “Neutralizer Remaining Amount (kg) [Unsolved]” may automatically be described by the management server 23 based on the management/process data 30 in the database 24.

The alarm history column 258 includes description columns “Alarm Occurrence Date and Time” and “Occurring Alarm”, together with a title “3. Alarm History”. The management server 23 automatically describes these description columns based on the management/process data 30 in the database 24.

The captain's signature column 250 includes a description column “Captain's Signature”. The captain reviews this “Ballast Process, Report (Detailed)” and signs in the description column “Captain's Signature”. This “Ballast Process, Report (Detailed)” is usable as the journal of the ship 4.

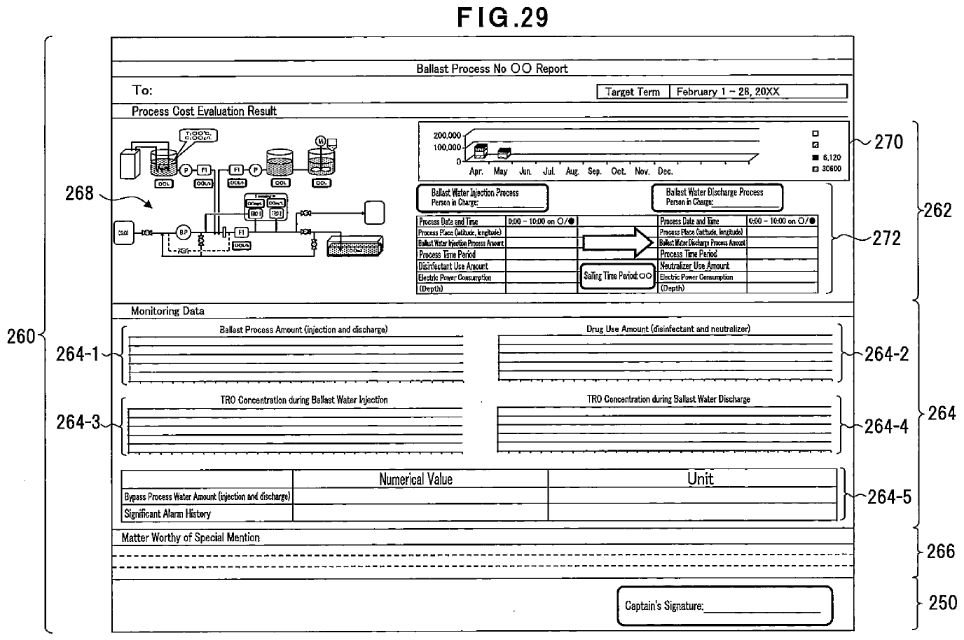

FIG. 29 is a diagram of another example of a reporting document. This reporting document 260 is an example of the reporting document prepared based on the management/process data 30, and includes a process cost evaluation column 262, a monitoring data column 264, a special description column 266, and the captain's signature column 250, together with a title “Ballast Process No Report”, an addressee description, and a target term description. The issuance of the report number is similar to that of the overview reporting document 242. The management server 23 inserts the issued report number between the title “Ballast Process No.” and “Report”, and stores the latest report number in the memory part 72. The management server 23 identifies a customer from the log-in ID and displays the name of the identified customer together with the addressee description.

In the left-side portion of the process cost evaluation column 262, an equipment configuration diagram 268 of the ballast water processing apparatus 8-1 is displayed. The equipment configuration diagram 268 includes the latest equipment data 11 relating to the ballast water processing apparatus 8-1. The equipment data 11 included in the equipment configuration diagram 268 is similar to the data displayed using the selection button in the display area 182-2 in the water process status screen 200 and therefore will not again be described.

In the upper-right portion of the process cost evaluation column 262, the cost associated with the operation of the ballast water processing apparatus 8-1 is displayed as a graph 270 of the monthly change. The cost associated with the operation is classified into a disinfectant cost, a neutralizer cost, an electric power cost, and the like, and is displayed using, for example, a stacked bar chart.

In the center-right portion and the lower-right portion of the process cost evaluation column 262, the series of water injection and discharge process results 272 from the ballast water injection process to the ballast water discharge process are displayed. The ballast water injection process result includes display items “Person in Charge”, “Process Date and Time”, “Process Place (latitude, longitude)”, “Ballast Water Injection Process Amount”, “Process Time Period”, “Disinfectant Use Amount”, “Electric Power Consumption”, and a display of “(Depth)” of the place of the ballast water injection process. The display items “Person in Charge”, “Process Date and Time”, “Process Place (latitude, longitude)”, “Ballast Water Injection Process Amount”, “Process Time Period”, and “(Depth)” of the place of the ballast water injection process are displayed similarly to the display items of the ballast water process column 246. The display item “Disinfectant Use Amount” is displayed similarly to the display item of the drug information column 256. The display item “Electric Power Consumption” has the electric power consumption needed for the ballast water injection process displayed therein. The ballast water discharge process result includes display items “Person in Charge”, “Process Date and Time”, “Process Place (latitude, longitude)”, “Ballast Water Discharge Process Amount”, “Process Time Period”, “Neutralizer Use Amount”, “Electric Power Consumption”, and a display of “(Depth)” of the place of the ballast water discharge process. The display items “Person in Charge”, “Process Date and Time”, “Process Place (latitude, longitude)”, “Ballast Water Discharge Process Amount”, “Process Time Period”, and “(Depth)” of the place of the ballast water discharge process are displayed similarly to the display items of the ballast water process column 246. The display item “Neutralizer Use Amount” is displayed similarly to the display item of the drug information column 256. The display item “Electric Power Consumption” has the electric power consumption needed for the ballast water discharge process displayed therein. The water injection and discharge process result 272 includes a “Sailing Time Period” display and this “Sailing Time Period” display has a time period displayed therein that is acquired by subtracting the process date and time of the ballast water injection process from the process date and time of the ballast water discharge process.

The monitoring data column 264 includes display columns 264-1, 264-2, 264-3, 264-4, and 264-5. The display column 264-1 is, for example, a display column “Ballast Process Amount (injection and discharge)”. The graph displayed in this display column 264-1 is a graph formed by combining the graph in the display column 254-1 and the graph in the display column 254-3 displayed in the detailed reporting document 252. The display column 264-2 is, for example, a display column “Drug Use Amount (disinfectant and neutralizer)” and is a graph whose axis of ordinate represents the use amount (L) of the drugs, whose axis of abscissa represents the process time period (h), and that displays, for example, the changes of the use amounts of the disinfectant and the neutralizer. The graph displayed in the display column 264-3 is the graph similar to the graph in the display column 254-2 displayed in the detailed reporting document 252, and the graph displayed in the display column 264-4 is the graph similar to the graph in the display column 254-4 displayed in the detailed reporting document 252. In the display column 264-5, the process result is described for the case where an unforeseen or exceptional intake or discharge of the ballast water occurs, corresponding to the miscellaneous item column 248 of the overview reporting document 242. In the display column 264-5, for example, the water injection amount and the water discharge amount of the bypass process water amount are described and the history of significant alarms is described. The bypass process water amount is the water amount used when the bypass process is executed without executing the disinfection process using the disinfectant and the neutralization process using the neutralizer, and the significant alarm is an alarm that causes any exceptional process such as the bypass process.

The reporting document 260 is usable as the journal of the ship 4. FIG. 22 to FIG. 29 respectively depict examples of the water process status screen 200, the trend display screen 220, the integral display screen 232, the management data display screen 234, the drug information display screen 236, the overview reporting document 242, the detailed reporting document 252, and the reporting document 260 for the ballast water processing apparatus 8-1. The screens and the reporting documents for the boiler apparatus 10-1 or the water desalination processing apparatus 12-1 only have to be displayed or printed in the same manner as that of these screens and the reporting documents for the ballast water processing apparatus 8-1.

### <Output Process for Management/Process Data for Control Apparatus 14-1>

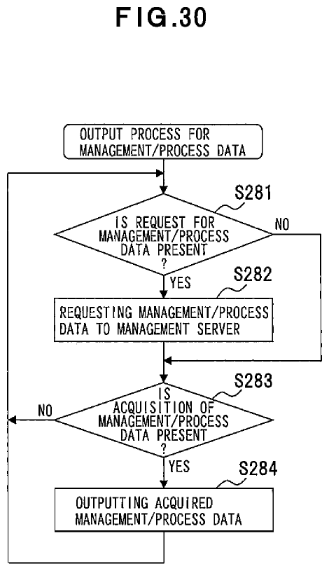

FIG. 30 depicts an example of a process procedure for an output process for the management/process data 30. This process procedure is an example of the equipment management method of the present invention. This process procedure is executed by execution of the equipment management program readably stored in the memory part 60 by the processor 58 of the control apparatus 14-1 and execution of the communication by the communicating apparatus 16-1. The processes are an example of the functions executed with the program or the management method. In FIG. 30, “step S” represents a processing stage.

The processor 58 determines whether any request for the management/process data 30 is present (step S281). As to the determination of the request for the management/process data 30, it is determined that the request for the management/process data 30 is present when an inputting is executed into, for example, the input part 64 to request the management/process data 30. When the processor 58 determines that the request for the management/process data 30 is present (YES of step S281), the processor 58 requests the management/process data 30 to the management server 23 through the communicating apparatus 16-1 (step S282). For example, when the input part 64 accepts an acquisition instruction for the water process status screen 200 by the user, the control apparatus 14-1 notifies the management server 23 of a transmission request for the water process status screen 200 through the communicating apparatus 16-1 based on the instruction accepted by the input part 64. When the processor 58 determines that any request for the management/process data 30 is not present (NO of step S281), the processor 58 does not execute step S282.

The management/process data 30 is transmitted to the control apparatus 14-1 through the communicating apparatus 16-1 by the request for the management/process data 30 or by an automatic process by the management server 23. For example, the requested water process status screen 200 is acquired from the management server 23. The processor 58 determines whether any acquisition of the management/process data 30 is present (step S283). When the processor 58 determines that the acquisition of the management/process data 30 is present (YES of step S283), the processor 58 analyzes and outputs the acquired management/process data 30 (step S284). For example, the control apparatus 14-1 displays the water process status screen 200 on the displaying part 62 based on the management/process data 30. The control apparatus 14-1 prints the water process status screen 200 using a printing apparatus connected to the I/O 66 based on the management/process data 30. When the processor 58 determines that any acquisition of the management/process data 30 is not present (NO of step S283), the processor 58 does not execute step S284. The processor 58 returns to step S281 and repeats step S281 to step S284.

### <Output Process for Management/Process Data 30 for Management Server 23>

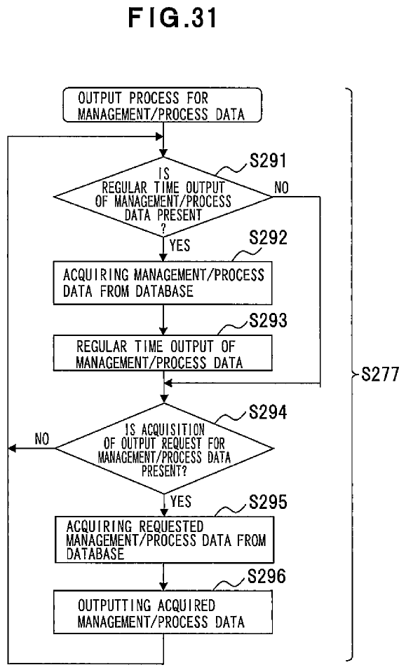

FIG. 31 depicts an example of a process procedure for an output process for the management/process data. This process procedure is an example of the equipment management method of the present invention. This process procedure is executed by execution of the equipment management program readably stored in the memory part 72 by the processor 70 of the management server 23. This process procedure is executed corresponding to, for example, the output process (step S277) for the management/process data depicted in FIG. 18. The processes are an example of the functions executed with the program or the management method. In FIG. 31, “step S” represents a processing stage.

The processor 70 determines whether any regular time output of the management/process data 30 is present (step S291). For the regular time output, the time is set for the transmission to be executed, for example, every one day (24 hours) and, for example, the overview reporting document 242 and the detailed reporting document 252 used as the journal are transmitted. When the processor 70 determines that the regular time output is present (YES of step S291), the processor 70 acquires the management/process data 30 such as the overview reporting document 242 and the detailed reporting document 252 from the database 24 (step S292) and executes the regular time output for the customers engaging in the ship (step S293). When the processor 70 determines that the regular time output is not present (NO of step S291), the processor 70 does not execute step S292 and step S293.

The processor 70 determines whether any acquisition of an output request for the management/process data 30 is present (step S294). When the processor 70 determines that the acquisition of the output request is present (YES of step S294), the processor 70 acquires the requested management/process data 30 from the database 24 based on the request (step S295) and outputs the acquired management/process data 30 to the requesting customer (step S296). When the processor 70 determines that any acquisition of an output request is not present (NO of step S294), the processor 70 does not execute step S295 and step S296. The processor 70 returns to step S291 and repeats step S291 to step S296.

In this manner, the management/process data 30 such as the analysis results, the diagnosis results, and the trend graphs produced by the management server 23 can be acquired by the ship 4, the manager, or the like and can be displayed and printed.

The ship 4 acquiring the management/process data 30 can refer to the management/process data 30 displayed on, for example, the displaying part 62, the person in charge of the pieces of water process equipment can adjust the drug solution injection amounts to the pieces of water process equipment, and the equipment load on the pieces of water process equipment can be adjusted.

### Effects of Example 2

(1) The effects described for Example 1 can be achieved.

(2) The management center 6, the ship 4, the ship owner, or the ship manager can analyze and diagnose the pieces of water process equipment and can display and print the trend graphs, based on the management/process data 30 stored in the database 24. Because the management server 23 acquires the latest equipment data 11 by radio, the analysis result, the diagnosis result, the trend graph, and the like reflecting the latest equipment status can be displayed and printed.

(3) The control apparatus 14-1 can acquire by radio the management/process data 30 produced by the management server 23 and can display or print the management/process data 30. Because of the intervention of the radio, the time period can be reduced that is necessary for the steps from the sampling of the water in the pieces of water process equipment to the displaying and printing of the trend graphs and the like based on the management/process data 30. The ship 4 can maintain the pieces of water process equipment each in a stable and optimal operation state by adjusting the drug solution injection amounts to the pieces of water process equipment and adjusting the equipment loads on the pieces of water process equipment, referring to the trend graphs and the like displayed on the displaying part 62 of the control apparatus 14-1.

(4) The data desired to be referred to can be relatively easily displayed or printed by the operation of the selection buttons and the switch buttons. For the operation of the drug replacement button 240, for example, a touch operation onto a touch panel, or a clicking operation by a mouse only has to be used. As to the various types of screen including the drug information display screen, updating of the equipment data 11 and the management/process data 30 can relatively easily be executed by the button operations in the display screen as the replacement end instruction using the drug replacement button 240.

(5) The output reporting document is usable as it is as the ballast water record book stipulated in the ballast water management convention and the journal, and the load of preparing these can be reduced.

### Modification Example

(1) Various modifications described in Example 1 can be made.

(2) The authentication is executed such that only the accesses by the users registered in the management server 23 are permitted by setting the log-in IDs and passwords in Examples while the present invention is not limited to this authentication. Access authority for the equipment data 11 on the specific water process equipment and the specific water quality analyzer 174 may be provided by correlating the ID and the password with the water process equipment and the water quality analyzer 174. With this configuration, only the management/process data 30 on the water process equipment can be acquired that is handled by the user corresponding to the ID, any viewing of the management/process data 30 on the water process equipment and the water quality analyzer 174 can be prohibited that are handled by other users, and the information security can thereby be further enhanced. The data may publicly be disclosed without executing any authentication by inputting the ID and the password.

(3) The equipment configuration diagram depicting the equipment configuration of the ballast water processing apparatus 8-1 is displayed in the display area 182-2 in the above Example while remote operation of the water process equipment may be enabled through this equipment configuration diagram. According to this configuration, the shipman reviewing the management/process data 30 provided by the management server 23 can execute the remotely operate the pieces of water process equipment through the control apparatus 14-1 that displays thereon the management/process data 30 provided by the management server 23 without moving to the operational panel of each of the pieces of water process equipment.

### Example 3

In Example 3, the equipment management system arranges the drugs used in the pieces of water process equipment such as the ballast water processing apparatus 8-1, the boiler apparatus 10-1, and the water desalination processing apparatus 12-1 of the ship 4. The equipment management system 2 of Example 3 includes the equipment management system 2 of Example 1 or the equipment management system 2 of Example 2. The content common to Example 1 or Example 2 will not again be described.

In Example 3, the management server 23 stores the travel information on the ship 4 in the database 24. The travel information on the ship 4 includes the sailing plan of the ship 4 and includes, for example, the anchorage site information on the ship 4 and staying term information on the anchorage site. The travel information only has to be acquired from, for example, the customers engaged in the ship. The management server 23 determines whether any of the drugs used in the pieces of water process equipment is insufficient. When the management server 23 determines that a drug is insufficient, the management server 23 refers to the travel information stored in the database 24, searches for the anchorage sites at which the ship 4 is scheduled to anchor, and arranges the drug at the anchorage site that is found by the search. The arrangement information on the drug is added to the management/process data 30 and is stored in the database 24. The ship 4 can refer to the management/process data 30 in the database 24 and can thereby learn the arrangement for the drug.

### <Arrangement Process for Drug>

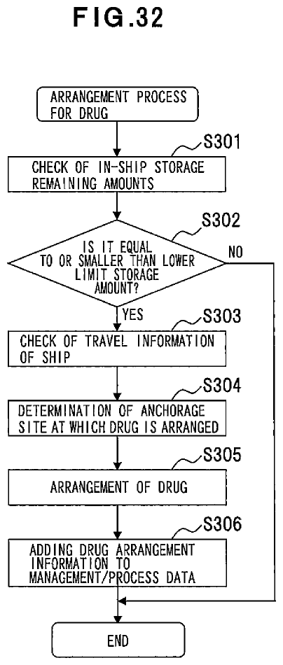

FIG. 32 depicts an example of a process procedure for an arrangement process for a drug. This process procedure is an example of the equipment management method of the present invention. This process procedure is executed by execution of the equipment management program readably stored in the memory part 72 by the processor 70 of the management server 23. This process is executed, for example, regularly such as, for example, every one day or every one week. The processes are an example of the functions executed with the program or the management method. In FIG. 32, “step S” represents a processing stage.

The processor 70 refers to the management/process data 30 and checks the in-ship storage remaining amounts of the drugs (step S301), and determines whether any in-ship storage remaining amount is equal to or smaller than the lower limit storage amount of the drug set in advance in the database 24 (step S302). When the processor 70 determines that the in-ship storage remaining amount is equal to or smaller than the lower limit storage amount (YES of step S302), the processor 70 checks the travel information of the ship 4 stored in the database 24 (step S303) and determines the anchorage site at which the drug is arranged (step S304). The anchorage site at which the drug is arranged is not limited to the anchorage site for the ship 4 to anchor next. For example, the date for the drug to run out may be estimated from the in-ship storage remaining amount of the drug and, when the drug seems to remain until the time of anchoring at the N-th anchorage site in the future, the drug may be arranged at the N-th anchorage site.

The processor 70 arranges the drug by, for example, issuing an order form to the determined anchorage site (step S305), adds the drug arrangement information to the management/process data 30 (step S306), and causes the process to come to an end. When the processor 70 determines that the in-ship storage remaining amount is not equal to or smaller than the lower limit storage amount (NO of step S302), the processor 70 does not execute step S303 to step S306 and causes the process to come to an end.

### Effects of Example 3

(1) The effects described in Example 1 or Example 2 can be achieved.

(2) Because the drugs necessary for the pieces of water process equipment are managed by the management server 23 and are arranged at the anchorage sites, the shipmen of the ship 4 do not need to manage and arrange the drugs and the work load on the shipmen is reduced. Because the drug arrangement information is stored in the management/process data 30, the pieces of information relating to the pieces of water process equipment of the ship 4 can collectively be recorded including the arrangement information on the drugs.

(3) When shortage of any of the drugs or equipment abnormality difficult to be repaired occurs on the sea, replenishment of the drug at the closest anchorage site or arrangement for the equipment repair occurs while these work steps are executed by the management center 6 and the arrangement load on the shipmen is reduced.

### Modification Example

(1) Various modifications described in Example 1 or Example 2 can be made.

(2) The management server 23 executes duties including the arrangement of the drug in the above Example while, for example, the management server 23 may generate an alarm urging the arrangement for the drug and an operator of the management server 23 confirming the alarm may arrange the drug.

(3) The drug is arranged in the above Example while management and arrangement of, for example, spare articles used for the pieces of water process equipment may be executed, and any abnormality of any of the pieces of water process equipment may be sensed and the repair or the maintenance of the water process equipment may be arranged.

As above, the most preferred embodiment and the like of the present invention have been described. The present invention is not limited by the above description. Various modifications and changes can be made thereto by those skilled in the art based on the gist of the present invention descried in the appended claims or disclosed herein. Not to mention, these modifications and changes are encompassed in the scope of the present invention.

### INDUSTRIAL APPLICABILITY

The present invention can provide the equipment data on pieces of water process equipment in a ship to the ship, the ship owner, or the ship manager, and is therefore useful.

### EXPLANATIONS OF LETTERS OR NUMBERS

2 equipment management system
4 ship
6 management center
8 ballast water process equipment
8-1 ballast water processing apparatus
8-2, 10-2, 12-2 control part
10 boiler equipment
10-1 boiler apparatus
11 equipment data
12 water desalination process equipment
12-1 water desalination processing apparatus
14, 22 processing part
14-1 control apparatus
14-2 storing part
16, 20 communicating part
16-1 communicating apparatus
18 public radio communication satellite
23 management server
24 database
26 communication equipment
27 communication network
28 ship/equipment data
30 management/process data
34 equipment information
36 ship code
38 ship name information
40 equipment code
42 equipment name information
44 water type name information
46 file name information
48 date information
50 time of day information
52 equipment data
54 sensor calibration date information
56 communicating part
58, 70 processor
60, 72 memory part
62 displaying part
64 input part
66, 74 I/O
68 communicating part
174 water quality analyzer
175 data analyzing apparatus

1. An equipment management system comprising:
a communicating part that acquires from a ship by radio notification information including one piece of state information, or two or more pieces of state information selected from pieces of state information on at least ballast water process equipment, boiler equipment, and water desalination process equipment loaded on the ship; and
a processing part that extracts the pieces of state information on the ballast water process equipment, the boiler equipment, or the water desalination process equipment from the notification information acquired from the ship, produces management information including drug information at least necessary for the ballast water process equipment, the boiler equipment, or the water desalination process equipment, and causes the communicating part to send out the management information to the ship sending out at least the notification information.

2. The equipment management system according to claim 1, wherein
the processing part causes the communicating part to send out the management information to an owner or a manager of the ship that sends out the notification information.

3. The equipment management system according to claim 1, wherein
the management information further includes reporting document information to be used for a journal information or a ballast process report.

4. The equipment management system according to claim 1, wherein
the processing part manages remaining amounts of drugs necessary for the pieces of equipment of the ballast water process equipment, the boiler equipment, and the water desalination process equipment and arranges any one of the drugs at an anchorage site of the ship in accordance with the remaining amount of the drug.

5. An equipment management system comprising:
a processing part that produces notification information including one piece of state information, or two or more pieces of state information selected from pieces of state information on at least ballast water process equipment, boiler equipment, and water desalination process equipment loaded on a ship; and
a communicating part that transmits by radio the notification information produced by the processing part.

6. The equipment management system according to claim 5, wherein
the communicating part receives by radio management information including drug information at least necessary for the ballast water process equipment, the boiler equipment, or the water desalination process equipment, and sends out the management information to the processing part, and wherein
the processing part presents the management information.

7. An equipment management program executed by a computer, the equipment management program for causing the computer to implement:
a function of producing notification information that includes one piece of state information, or two or more pieces of state information selected from pieces of state information on at least ballast water process equipment, boiler equipment, and water desalination process equipment loaded on a ship;
a function of extracting the pieces of state information on the ballast water process equipment, the boiler equipment, or the water desalination process equipment from the notification information and producing management information that includes drug information necessary for the ballast water process equipment, the boiler equipment, or the water desalination process equipment; and
a function of sending out the notification information or the management information.

8. An equipment management method comprising:
producing notification information that includes one piece of state information, or two or more pieces of state information selected from pieces of state information on at least ballast water process equipment, boiler equipment, and water desalination process equipment loaded on a ship;
extracting the pieces of state information on the ballast water process equipment, the boiler equipment, or the water desalination process equipment from the notification information and producing management information that includes drug information necessary for the ballast water process equipment, the boiler equipment, or the water desalination process equipment; and
sending out the notification information or the management information.
